(19) United States
(12) Patent Application Publication
(10) Pub. No.: US 2002/0143471 A1
(43) Pub. Date: Oct. 3, 2002

(54) METHOD OF PREDICTING AND GENERATING DNA SEQUENCES AND APPARATUS FOR CARRYING OUT THE METHOD

(76) Inventor: HARRY MONTAGUE, BROOKLINE, MA (US)

Correspondence Address: SUGHRUE MION ZINN MACPEAK & SEAS PLLC, 1010 EL CAMINO REAL, SUITE 360, MENLO PARK, CA, 94025

(21) Appl. No.: 09/444,981

(22) Filed: Nov. 22, 1999

Related U.S. Application Data
(63) Continuation-in-part of application No. 08/949,927, filed on Oct. 14, 1997, now abandoned.
Application No. 08/949,927 is a continuation-in-part of application No. 08/455,328, filed on May 31, 1995, now abandoned.
Application No. 08/455,328 is a continuation-in-part of application No. 08/366,929, filed on Dec. 30, 1994, now abandoned.

(51) Int. Cl. G06F 19/00; C12Q 1/68

(52) U.S. Cl. 702/20; 435/6

(57) ABSTRACT

The inventive method woks by assigning complex numbers to a rearranged format of the genetic code, this format using the last codon letters as the organizing force. These numbers verify the genetic code mathematically and are tied to the behavior of the hydrogen atoms on the hydrogen bridge between DNA strands. This behavior is explained in part by a formula for modeling the wave behavior of the DNA molecule and the holographic behavior of the DNA molecule as an information-storing material.

### REFERENCE TO RELATED APPLICATIONS

This application is a continuation-in-part of application Ser. No. 08/949,927, filed Oct. 14, 1997, which is a continuation-in-part of application Ser. No. 08/455,328, filed May 31, 1995, now abandoned, which is a continuation-in-part of application Ser. No. 08/366,929, filed Dec. 30, 1994, now abandoned. All of these applications are incorporated by reference into the present application.

### BACKGROUND OF THE INVENTION

The present invention relates to a method of modeling sequences of dioxyribonucleic acid (DNA,) the building blocks of life. The method pertains in particular to a method of generating and/or predicting DNA sequences in accordance with the inventive modeling method. The invention also relates to an apparatus for taking advantage of a relationship which the inventor has identified between the complex number system and the codons from which DNA molecules are formed. Exploitation of this relationship reveals a computational aspect to molecular biology, which can be explained in part based on holographic principles that reflect the behavior of electrons along the hydrogen bridges and the helical strands of DNA.

As is believed will be apparent from a review of the detailed discussion herein, the concepts described are not strongly biotechnology-oriented. Rather, they represent the results of the inventor's observations and analysis of the structure of codons, as related to the structure of DNA, and what appears to be a mathematical relationship which can be exploited advantageously in generating and predicting DNA sequences.

Scientists have experimented with DNA for years, in an attempt, inter alia, to understand its origins, and to understand how codons, which are subelements of DNA, combine to form particular DNA sequences. The hydrogen bridge between the helical strands of DNA has been investigated exhaustively, and distances along the bridge identified. However, while theories have been put forth regarding prospects for using DNA in computing applications, so far as the present inventor is aware, no one previously has been able to take any particular advantage of this knowledge, especially as it might be applied to predicting DNA sequences, or ultimately to generating such sequences.

A November 1994 article in The New York Times, and a May 1995 article in Newsweek discuss the use of DNA to perform intricate computations more efficiently than presently is believed possible with conventional, non-organic computing devices (i.e. desktop or mainframe computers). The potential use of DNA as a kind of computer has piqued the curiosity of scientists for exploring other ways in which DNA might be exploited. However, so far as the present inventor is aware, no one has identified, with the precision disclosed herein, the fundamental relationship between DNA molecules and the complex numbers.

The identification of varying distances along the hydrogen bridge between strands of DNA has attracted the inventor's curiosity for many years, leading to a comprehensive mathematical study in an attempt to relate these distances in some systematic way. The present invention is the result of these efforts.

### SUMMARY OF THE INVENTION

The amino acid complex conjugate numbering system in the patent application can be applied usefully, with the aid of a computer, in solving sequencing problems. As a very simple example, assign the conjugate numbers to the amino acids as shown herein. Then take a known sequence of amino acids and substitute the conjugate numbers. Program the computer to average the sum of the numbers over the desired sequence spans of, say, 1000, 100, and 10 codons. When the average of the numbers drops, it indicates that the selection of amino acids is moving up to smaller conjugate numbers, up the residue hydrophobicity scale of amino acids to higher values for free energy of transfer (see Principles of Biochemistry, Table 1, p. 278). For a more complex analysis, the computer can program both the amino acid series with the accompanying nucleotide letter series. Then substitute the conjugate numbers and their component integers which are assigned to each position of the codons. This would help in analyzing changes of the individual letters as it relates to changes of the amino acid characteristics. This type of very simple computer programming, using the numbers provided herein, is useful in amino acid sequencing analysis.

The present application describes a system that mathematically proves the reduction of the 64 letter nucleotide down to the 20 amino acids. It also describes a system that shows the organizational importance of the last letter of the codon, which has been thought to be redundant and mostly meaningless. A further feature of the invention is a system that, while explaining some known DNA molecular processes, also provides both an individual and a comprehensive numbering process as noted above. Table A shows the genetic code codon letter combinations organized around the last letters, the matching amino acids, the matching complex conjugate numbers and the matching (x) and (y) components numbers that work with the codon letters, all in identical formats. From the four part Table A, as stated before, come the mathematical numbers for the proof of the genetic code, the numbers for labeling each letter of the code. From these numbers on this table comes the ability to program a computer to test and enhance the DNA sequencing process.

In view of the foregoing, it is one object of the present invention to take advantage of a fundamental relationship between the complex numbers and the codons of DNA so as to enable prediction of DNA sequences and, hence, their generation based on assemblies of codons, which are the basic elements of DNA.

The standard genetic code format can be rearranged and organized around the last letters of each codon. The above complex numbers can be assigned to the codons in this rearranged format in a logical manner.

The inventive prediction method is based on an ability to map the DNA building blocks to complex numbers (a complex number having real and imaginary parts). The real and imaginary components of complex numbers can be plotted on orthogonal axes, the angle of intersection of these axes being wholly analogous to the perpendicular nature of the hydrogen bridge relative to DNA strands.

In accordance with the inventive method, then, each amino acid of the genetic code is assigned a conjugate number which is derived from a special complex number table. The code numbers on this two dimensional table are projected onto a single sloping line. The slope of this line and the spacing of the code numbers are comparable to a single line representing one chain of the DNA molecule.

The amino acids, whether in the two dimensional complex number form or in the single dimensional line form can be assigned to the side chains of the DNA molecule, the RNA molecule transferring this information, or the protein molecule-genetic code receiving this information. The circumferential side chain of the DNA molecule rises with a slope of 29°. The fold line of the two dimensional complex number table housing all the information of the genetic code rises with a slope of 29°. The side chains have a circumferential tangent point where the bases are joined and the conjugate numbers table has a starting axis point. If these two points are lined up, one can imagine the conjugate number table being wrapped around the DNA molecule so that their sloping lines coincide and the table's information can then be squeezed down to one codon segment of the chain. Stated in the simplest terms, all the information of the genetic code can be transferred to each codon using these sloping lines.

The mathematical proof of the genetic code requires the entire two dimensional format with both the real and imaginary components of complex numbers. But each chain of the DNA molecule can use only real numbers either the whole number conjugates, 2, 5, 8, 10, etc. formed from the sum of x²+y², or the component numbers (x) and (y) extracted from the complex numbers (x+yi)(x−yi). The component numbers (x) and (y) are tied to the amino acids, but can be used in place of the first, second, and third letters of the codon. The letter order of the DNA bases is transmitted to the side chain. Consequently, from the amino acid sloping line series, the protein forming system pulls, in any order, the individual amino acids that it wants, and places them into the correct corresponding order to match the DNA bases series.

The DNA molecule, with its amino acids and hydrogen bridges, uses waves which have certain wave characteristics. These wave characteristics can be put in a formula that ties the hydrogen bridges to the side chains to a uniform wave. The uniform wave characteristics are required in the wave holographic process used by the DNA molecule. The DNA molecule uses a holographic process to store the base information on its outer chain.

The mathematics in this wave characteristic formula can be used to form base numbers which can be put in a computer. These base numbers can be divided into three areas: the application of conjugate numbers to the genetic code; the use of 1 to 8 integers at each step of the DNA ladder; and the above systems altered at each step for the selection of the for different nucleotide bases. First, each amino acid is translated into a conjugate number which is represented by wave patterns on the outer chain of the DNA molecule. In addition to these wave patterns, each amino acid has interference fringe area caused by a diffraction pattern coming from the hydrogen bridge openings onto a screen on each codon position of the ladder. The three hydrogen bridge gaps form one combined diffraction pattern of the mound which matches the positions of the conjugate number taken from the complex number diagram. This diagram is squeezed down so it can fit onto each of the codons of the DNA ladder. Second, pick the integers 1 to 8 for x and y in the conjugate formula (x+yi)(x−yi) that matches the conjugate number selected. These integers can be applied to the wave characteristic formula that ties the hydrogen bridge waves, to the side chains waves. These wave patterns should be applied to the wave peaks effected by the conjugate number diffraction mounds. The integers are used with the x, +iy, and −iy on the combined first, second, and third steps of each codon, and together they reflect the conjugate selection for each codon. Third, the screen used on the outer chain has three multiple layers, each receiving the diffraction pattern from the hydrogen bridge. Each nucleotide step has one of four possible code letter selections, A, T, C, or G, which slightly tilt the hydrogen bridge at varying angles to the outside chain. In a holographic manner this change of angle causes the conjugate mounds to be pulled to one side or the other on each of the layers of the outer chain screen. These changes, when read by a wave traveling along the outer chain or by the outside RNA molecule reading the code, not only appear to be different in a holographic way, but actually are different. Thus, all three of the systems are tied together and mathematically consistent with each other. The outer chain is constantly changing object waves which are then compared with their new uniform waves and matched to the object waves coming from the hydrogen bridge. The ability to have uniform length waves on the outside chain which are compared to the hydrogen bridge is the key to this whole holographic process.

When the wave length positions of all of the component parts are set to conform to the amino acid selection this completes the basic system used by the DNA molecule. When the positions are set, then so is the amount of energy or wave amplitude.

### BRIEF DESCRIPTION OF THE DRAWINGS

The foregoing and other objects and features of the invention will become more apparent when taken in conjunction with the accompanying drawings, in which:

Table A is a table with the genetic code rearranged by last letter;

TABLE A

| I | | | | | | | | II | | | | | | | |
|---|---|---|---|---|---|---|---|---|---|---|---|---|---|---|---|
| 8 + 1 − 1 | 8 + 2 − 2 | 8 + 3 − 3 | 8 + 4 − 4 | 8 + 5 − 5 | 8 + 6 − 6 | 8 + 7 − 7 | 8 + 8 − 8 | 65₁ | 68₂ | 73₃ | 80₄ | 89₅ | 100₆ | 113 | 128 |
| | 7 + 1 − 1 | 7 + 2 − 2 | 7 + 3 − 3 | 7 + 4 − 4 | 7 + 5 − 5 | 7 + 6 − 6 | 7 + 7 − 7 | | 50 | 53 | 58 | 65 | 74 | 85 | 98 |
| | | 6 + 1 − 1 | 6 + 2 − 2 | 6 + 3 − 3 | 6 + 4 − 4 | 6 + 5 − 5 | 6 + 6 − 6 | | | 37 | 40 | 45 | 52 | 61 | 72 |
| | | | 5 + 1 − 1 | 5 + 2 − 2 | 5 + 3 − 3 | 5 + 4 − 4 | 5 + 5 − 5 | | | | 26 | 29 | 34 | 41 | 50 |
| | | | | 4 + 1 − 1 | 4 + 2 − 2 | 4 + 3 − 3 | 4 + 4 − 4 | | | | | 17 | 20 | 25 | 32 |
| | | | | | 3 + 1 − 1 | 3 + 2 − 2 | 3 + 3 − 3 | | | | | | 10 | 13 | 18 |
| | | | | | | 2 + 1 − 1 | 2 + 2 − 2 | | | | | | | 5 | 8 |
| | | | | | | | 1 + 1 − 1 | | | | | | | | 2 |

| | |
|---|---|
| III | IV |

| | | | | | | | | | | | | | | | |
|---|---|---|---|---|---|---|---|---|---|---|---|---|---|---|---|
| | | | | | | | | 2. U | A | A | U | U | U | A | A |
| | | | | | | | | 1. C | G | C | C | C | C | G | G |
| PHE | ARG | ARG | ASP | CYS | ASN | GLN | STOP | UU | CG | AG | GA | UG | AA | CA | UG |
| | LEU | PRO | TYR | ARG | HIS | GLU | MET | | UU | CC | UA | CG | CA | GA | AU |
| | | SER | SER | THR | PRO | LYS | ? | | | UC | AG | AC | CC | AA | UA |
| | | | LEU | ILEU | SER | THR | TRPP | | | | CU | AU | UC | AC | UG |
| | | | | VAL | VAL | LEU | ILEU | | | | | GU | GU | CU | AU |
| | | | | | ALA | ALA | ? | | | | | | GC | GC | UA |
| | | | | | | GLY | GLY | | | | | | | GG | GG |
| | | | | | | | GLY | | | | | | | | GG |

Table B is a table with integers added to conjugate numbers;

TABLE B
SUPPLEMENT SHOWING COMBINED NUMBERS IN CORRECT
FORMAT

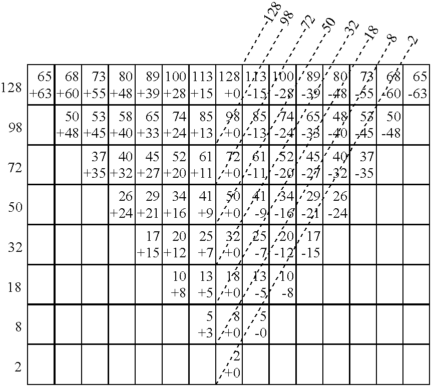

Table C is a table with summation and conjugate numbers;

TABLE C

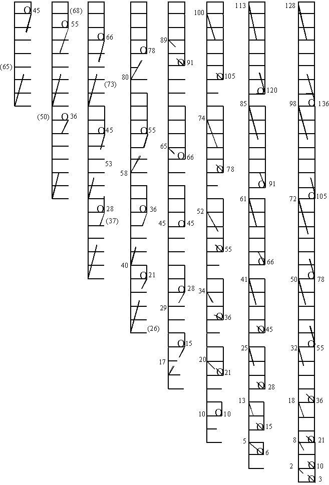

FIG. 1 is a graph depicting mapping of complex numbers onto orthogonal axes;

FIG. 2 is a table depicting a relationship between complex conjugates and codons in accordance with the present invention;

FIGS. 3-5 are graphs joining numbers from among complex conjugates;

FIG. 6 is a graph mapping complex conjugates onto comparative points;

FIGS. 7, 7A, 7B, and 7C are extended mappings of complex conjugates onto a horizontal axis, with spacings between conjugates showing relationships among conjugate pairings;

FIG. 8 is a table showing conventional identifications of codons;

FIG. 9 is a table showing a conversion of codons to complex conjugates;

FIG. 10 is a diagram showing the integer numbers on the hydrogen bridge and side chain;

FIG. 11 is a table showing complex conjugates grouped in a simplified relationship;

FIGS. 14 and 14A are depictions of the formation of a cycloid;

FIG. 15 is a spread-apart representation of a pendulum bob;

FIGS. 16 and 17 are graphs showing the relationship between sines and cosines;

FIGS. 18 and 19 are graphs showing the formation of a cycloid;

FIGS. 20-24 and 26-27 are graphs showing a relationship between a cycloid and a sine curve;

FIG. 28 is a representation of a relationship between a cycloid path and sine and cosine curves;

FIG. 29 is a representation of formation of particles at the start, middle, and end of a wave cycle;

FIGS. 30-32 are a graph of a spiral showing successive distances;

FIG. 38 is a conceptual cross section through a DNA molecule showing a relationship between reference chords and distances along the DNA chain and the molecular position of the bases; and

FIG. 40 is a graph showing the relationship between a Mobius strip molecular position of the bases and distances along a DNA chain.

### DETAILED DESCRIPTION OF A PREFERRED EMBODIMENT

As part of the background for understanding the present invention, it is useful to think of an equation having the DNA molecule on one side, and the amino acid molecules of the genetic code on the other. In between the two are the transcription and messenger RNA molecules that close the gap and equate these two types of molecules. The double helix of the DNA molecule as a twisted ladder is a very unusual molecular shape, with its sides and rung components perpendicular to each other.

As another useful background construct, it is helpful to think of the amino acid molecules as governed by a set of rules defined by a special set of complex numbers. Complex numbers are very unusual in that, when diagrammed, they are formed of components which are perpendicular to each other (i.e. the real and imaginary components,) as are the rungs and sides of the DNA molecule.

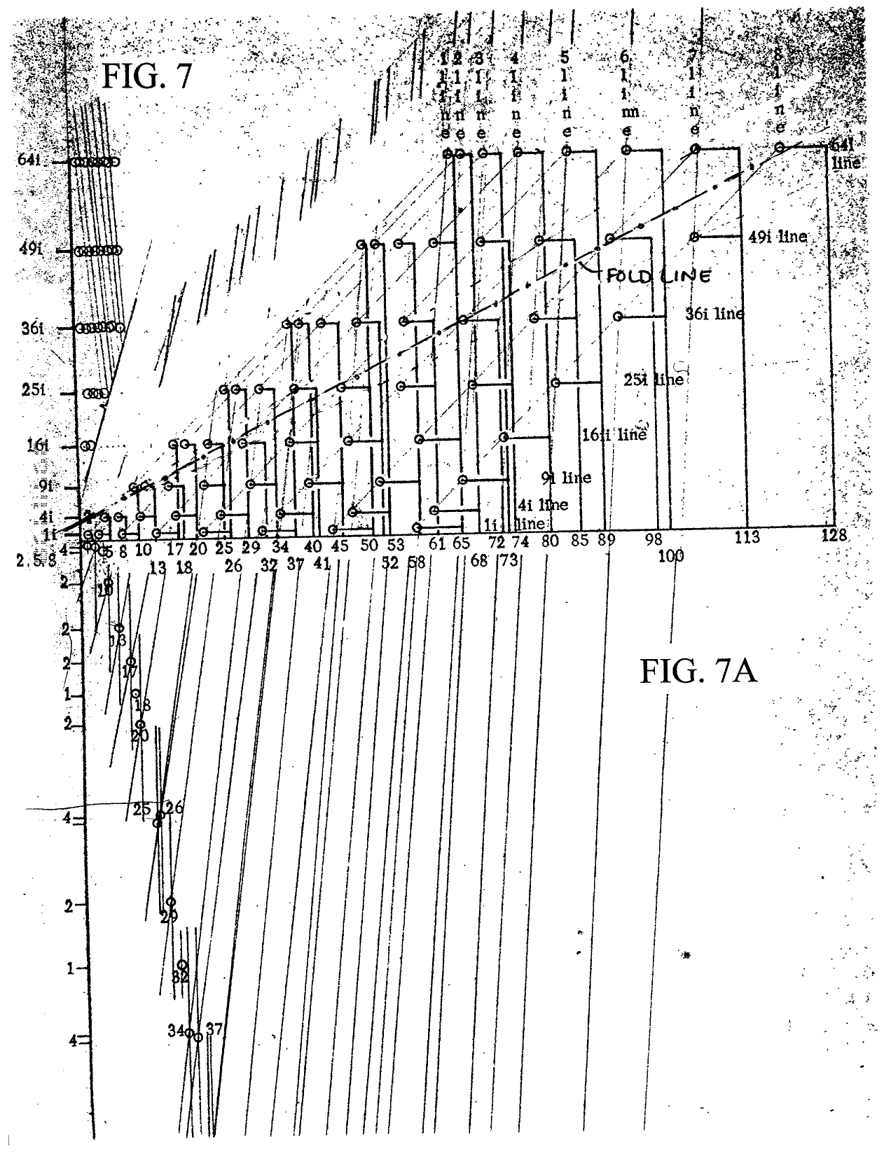

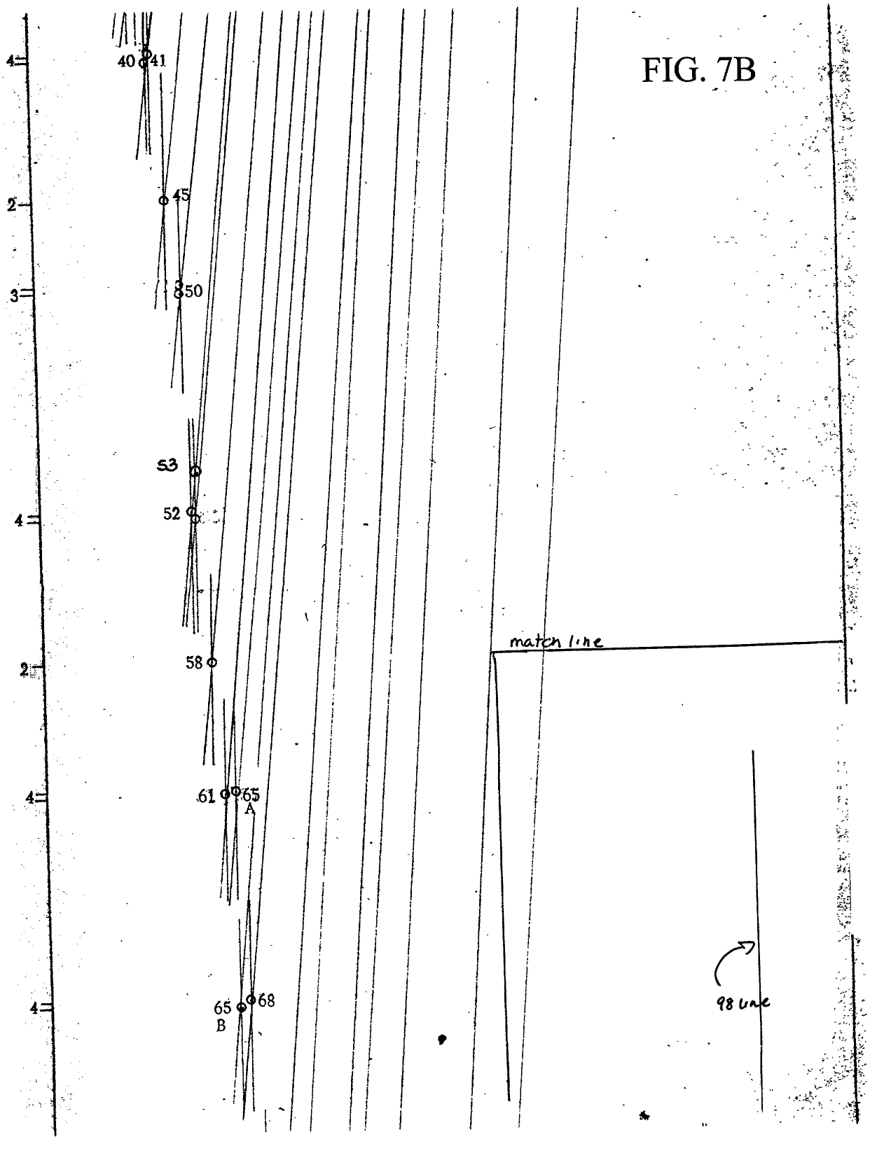

Using complex numbers to describe the DNA molecule turns out to be a perfect match—a match of number shapes to real shapes. The inventor has discovered that complex numbers can be used to define the genetic code of the amino acids. A key aspect of the investigation that led to this discovery focused on addressing the issue of what ties the two sides of the DNA molecule-amino acid equation together. The inventor has found the answer: the complex numbers of the code can be transcribed onto one single line. This line, shown dot-dash in FIG. 7, then can be used to form the helix lines of the sides of the DNA molecule. The pitch of this single line, holding all the code combinations as determined by the complex number diagram, is identical to the pitch of the helix line of the DNA molecule.

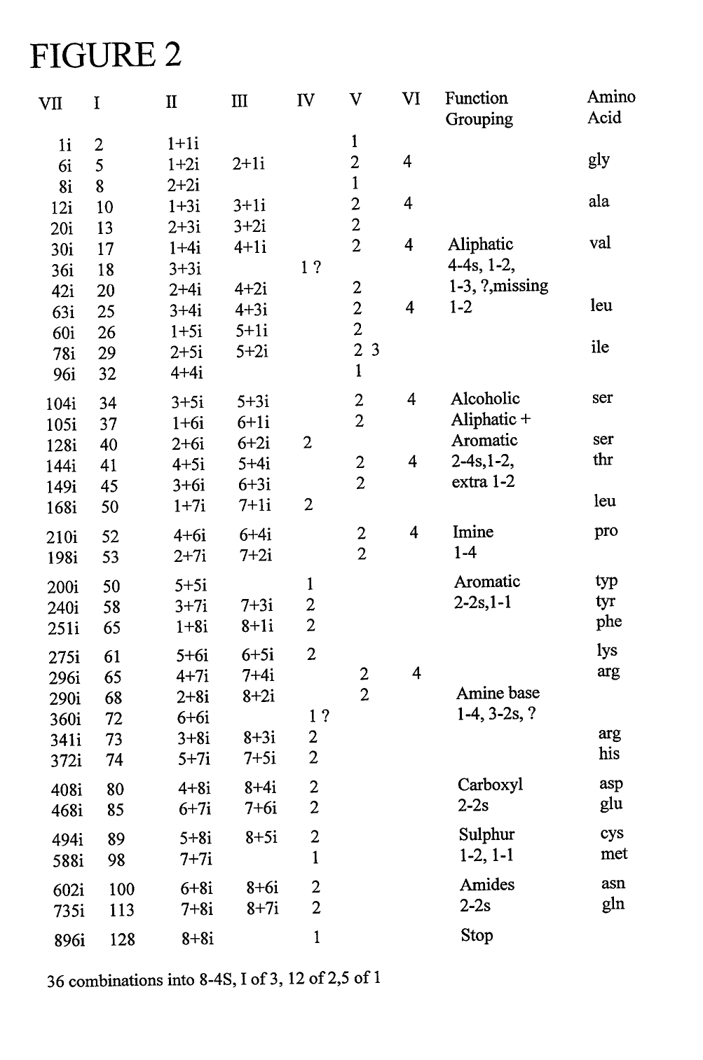

FIG. 7, housing this single line, shows the special complex number diagram which is used in association with the genetic code of life. Each amino acid of the genetic code can be assigned a complex conjugate code number, as shown in FIG. 2. This matching of numbers to the amino acid is based on three factors. The first is the number of codon combinations assigned to each amino acid. One codon combination, AUG, is assigned to the Methionine amino acid; two such combinations, UGU and UGC, are assigned to Cysteine; and so on. The second factor is the known genetic function grouping of the acids, coupled with a conjugate number sequence. The Aliphatic type amino acids use the first group of conjugate numbers from 2 to 32; the Alcoholic type amino acids use the conjugate numbers from 34 to 50; and so on. With the exception of the position of one group of the Leucine amino acid, all the other amino acids seem to fall in perfect order based on the conjugate sequence. The third factor is known hydrophobicity, coupled with a conjugate number sequence, as shown in FIG. 2.

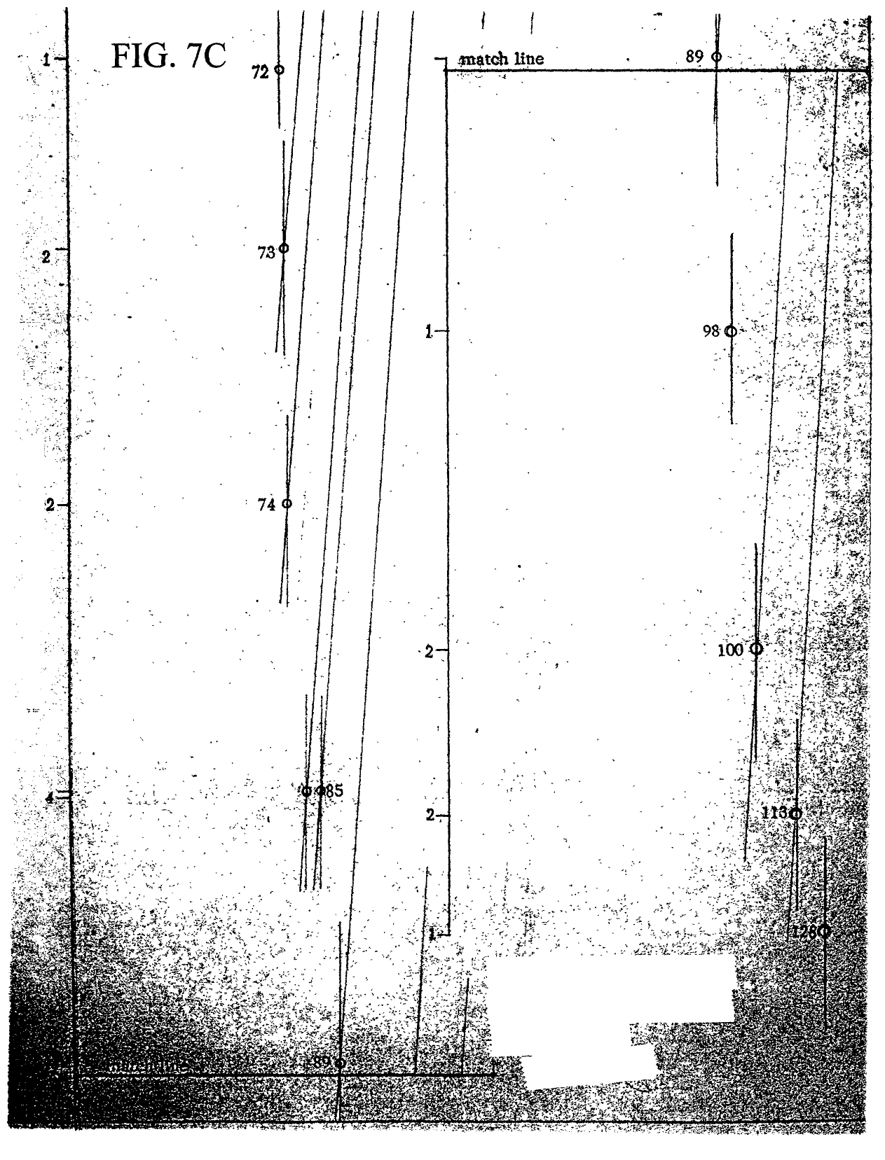

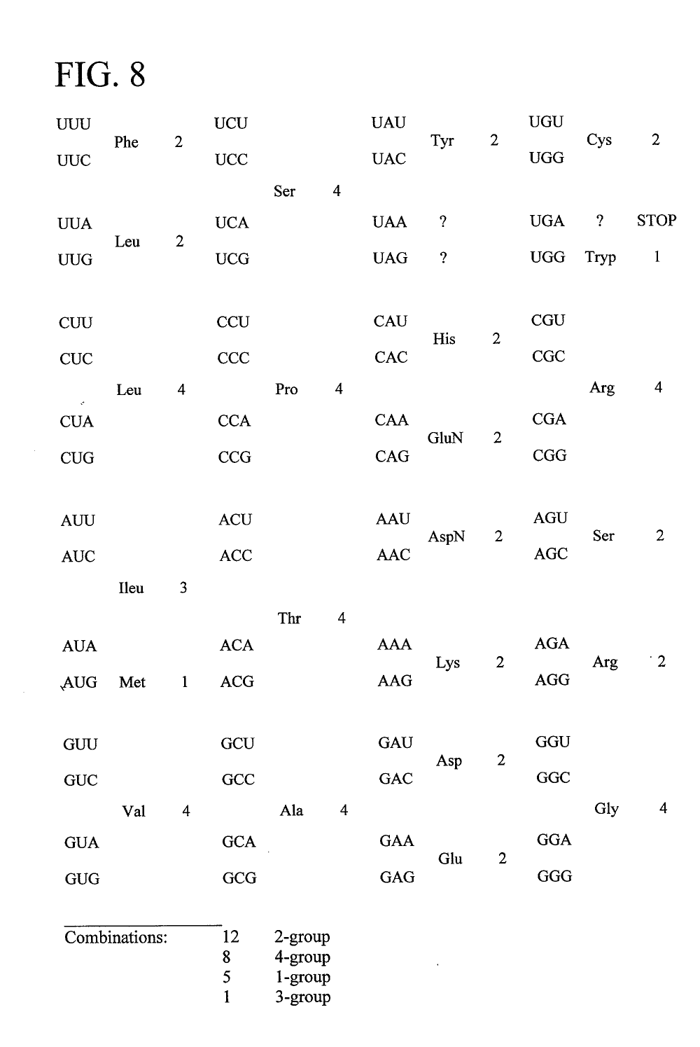

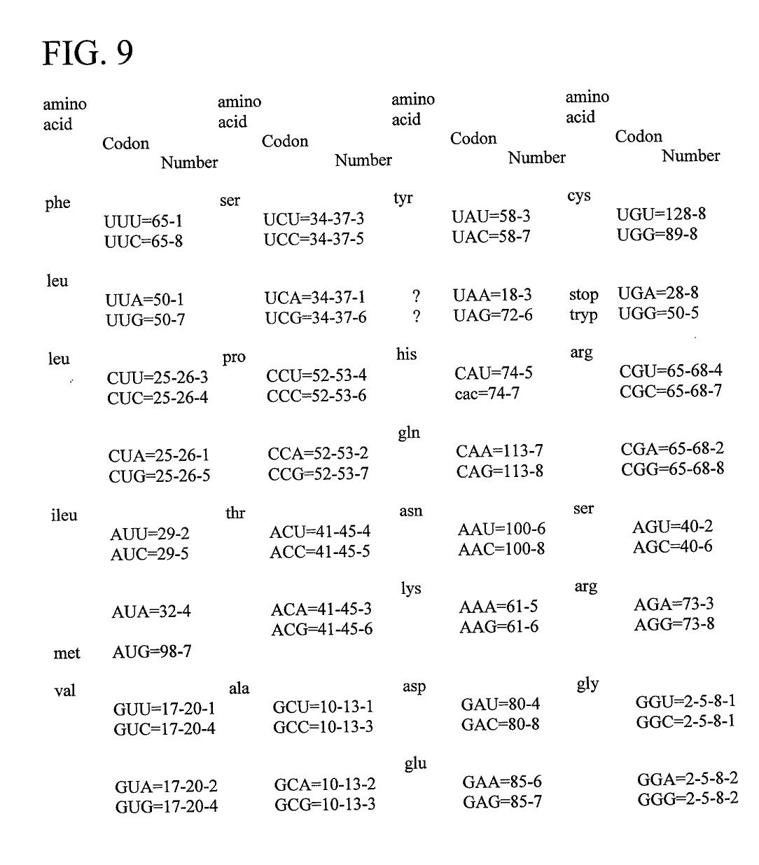

FIG. 8, showing the accepted genetic code, converts the amino acids to codon letter combinations. As a result, the complex number conjugates can be broken into their component parts, and each component assigned to each codon letter combination, as shown in FIG. 9. In nature, certain letter combinations are very prevalent, while others are very rare. This information, along with other factors, is worked into the assigning process.

The use of the conjugate numbers in place of the letter combinations permits the relation of the DNA molecule step positions to the derived positions of the amino acid of the FIG. 7 diagram. As with the chemical valence Figures, whose atomic rings help explain why the elements behave in a particular manner, using the amino acids' relative sequence positions will provide useful knowledge about the origin of the genetic code and DNA molecule base sequences, and why the amino acids function in a particular manner. Finally, there is a system built into the amino acid diagram which, when used with the DNA molecule, picks only one codon at a time, permitting completely random selection of codon sequences.

As has been shown by Dr. Leonard Adleman at the University of Southern California, as discussed in the above-mentioned articles in The New York Times and Newsweek, DNA molecules can work as high performance computers. Complex numbers are the comprehensive numbering system in mathematics. It can be said that the DNA molecule has the mathematical ability to work in a more multi-dimensional manner than suggested by an ordinary step by step sequence. Thus, the complex number sequence will be useful in future computer work involving the DNA molecule.

The Genetic Code

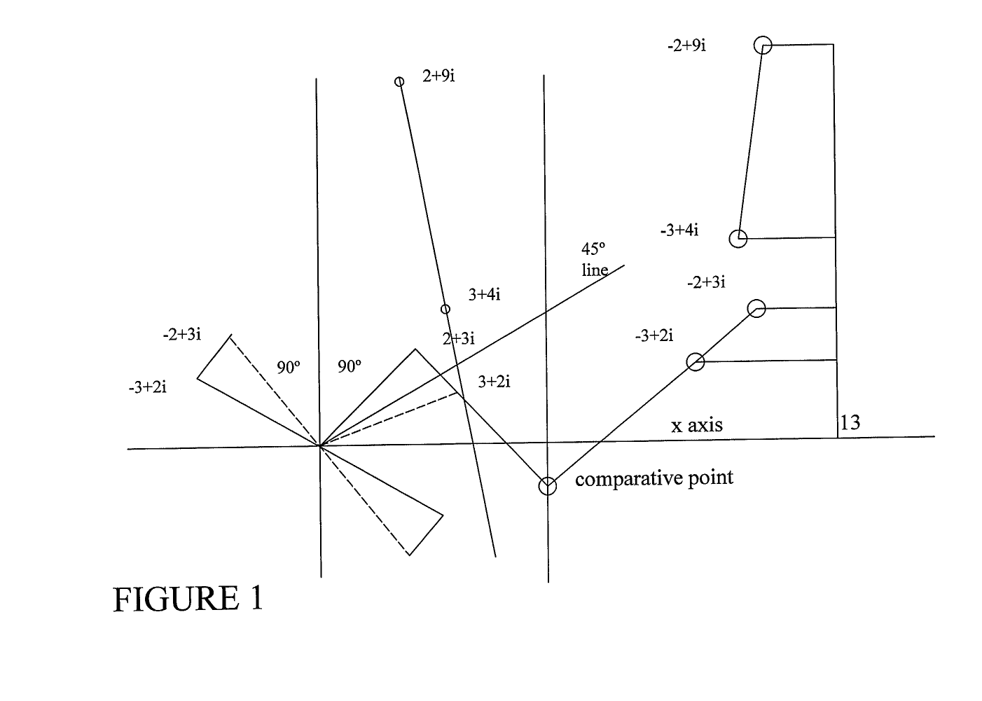

The best way to understand complex numbers and the joining of real and imaginary numbers, is a diagram showing the system for putting them together. FIG. 1 shows a complex number graph. The real number component of the complex number is plotted along the horizontal line, and the imaginary (i) component is plotted along the vertical line, the center point being zero. The complex number −3+2i is in the top left hand quadrant; 3+2i is in the top right quadrant; 3−2i is in the bottom right quadrant; and −3−2i, if it were shown, would be in the bottom left quadrant. The points −2+3i, 2+3i, and 2−3i similarly are shown in the same three respective quadrants. FIG. 1 shows a plot of these three arbitrary points. Using the horizontal axis as a center line, 3+2i and 2+3i are the set of symmetrical points of 3−2i and 2−3i about this line. They are shown plotted above the zero point on the imaginary vertical axis, thus the positive i values. Similarly, using the vertical axis as a center line, −3+2i and −2+3i are the set of points which are symmetrical to 3+2i and 2+3i.

As shown in FIG. 1, to go from 2−3i up to 3+2i, it is necessary to rotate counterclockwise by 90°. To rotate counterclockwise by 90° and plot the point (3+2i), simply multiply the original number by i; to turn clockwise, multiply the number by −i. (2−3i)(i)=(2) (i)−(3i)(i)=(2i+3) or (3+2i). The 90° separated points, −2+3i, 3+2i, and 2−3i, are joined together with a dotted line, just as the second group, −3+2i, 2+3i, and 3−2i are joined with a solid line. To change 3+2i in the top right quadrant to its complex conjugate, 3−2i, in the lower right quadrant, change the sign of the imaginary component from +2i to −2i. Complex conjugates multiplied together always equal a real number, e.g. (3+2i)(3−2i)=+9−6i+6i−(−4)=9+4=13. Also, it does not matter if the complex conjugate is −3+2i or −3−2i; they still multiply out to be the same real number: 9+4, or 13.

Now, take a new axis, located at the real number point 13 on the horizontal line in FIG. 1, which shows the points −2+3i and −3+2i plotted correctly to the left of the new axis, since their real number components are now negative. Since both −2+3i and 2+3i equal the real number 13 when multiplied by their respective complex conjugates, the fact that a number changes from +2 to −2 does not affect the answer, as mentioned before. Halfway between the old zero axis and the new axis is a vertical line on which there is a point labeled the “comparative point”. Now the set of four points lie symmetrically about this vertical comparative axis. Lines are drawn connecting the paired points between the stationary 2+3i and 3+2i and the moving −2+3i and −3+2i, lines are extended down through the x axis to the comparative point, labeled as such on FIG. 1. As will be seen later, by forming these comparative points and extending the real components of the complex numbers to the right along the real axis, the effect actually is to square the real number component.

To form the complex conjugate number chart, the imaginary components of the complex numbers are squared. Thus the first pair, 2+3i, and 3+2i become 2+9i and 3+4i, and the second pair −2+3i, −3+2i, become −2+9i and −3+4i. These four points again are plot and the new axis. The connecting lines are drawn; if they had been extended all the way out to intersect each other, they would have formed new comparative points. What has been done here is to draw a sample complex number, and square its imaginary component. This new comparative point, derived from the original pair, is one of the points used to form the mathematical comparison to the genetic code. As will be seen later, there also is an additional grouping of some of the numbers to form the code, that grouping being dependent on viewing the comparative points from a different perspective.

Looking now at the other complex number pairs that form the rest of the comparative points, FIG. 2, column I shows a complete list of these pairs along with their conjugate number multiples in columns II and III. (13 was the conjugate number multiple used above.) To simplify things, the negative sign on the real numbers has been dropped in FIG. 2. It also shows those complex numbers that do not pair up, where the real component and the imaginary component of the complex number have the same magnitude, e.g. −1+1i, −2+2i, etc. There are eight of these combinations, and 28 paired combinations, for a total of 36 groups. The list is in numerical order from the smallest number of −1+1i, with a conjugate multiple number of 2, up to the largest number −8+8i, with a conjugate multiple number of 128. Columns IV, V, and VI in FIG. 2 are the additional groupings of these pairs and single numbers into the single, paired, triple, and quadruple sets which will be explained later.

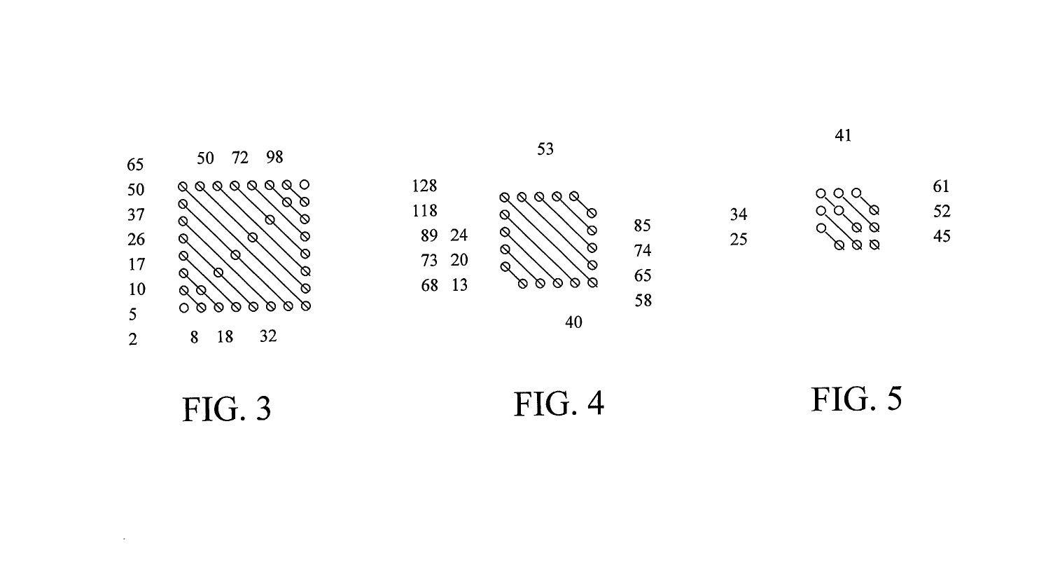

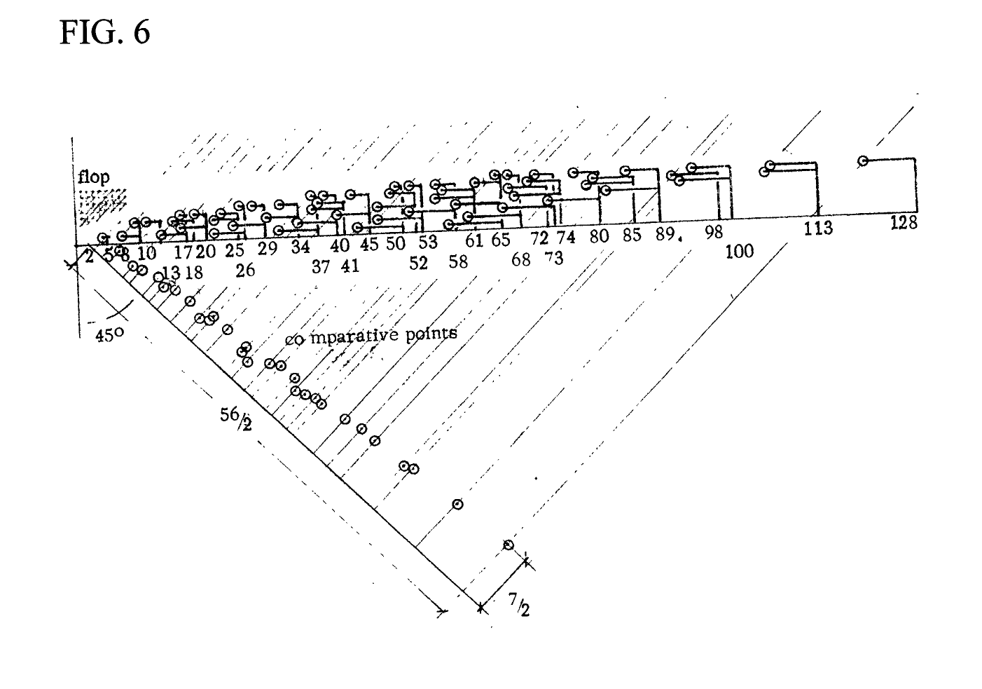

FIGS. 3, 4, and 5 show all the paired numbers connected by lines, with the single unpaired points shown in FIG. 3 along with the outside pairs. FIG. 4 shows the intermediate pairs; finally, FIG. 5 shows the inside pairs. The division of the number pairs into FIGS. 3, 4, and 5 is completely arbitrary, and is done in this case only to enable visualization of where all the pairs are. All the lines connecting these number pairs could be extended down and to the right as they were in FIG. 1. A sample extension is shown on each figure. Now, all these complex numbers are put back into an 8×8 block and are flip-flopped to put them into the −x quadrant. Each number is slid along the x axis over to the appropriate conjugate multiple point that forms the new zero axis line. At this point, it should be recalled that, in FIG. 1, the sample −3+2i and −2+3i pair was shown as moved, using 13 as the point for the new vertical zero axis line. In FIG. 6, all the numbers have been re-plotted off their conjugate multiple points, and appear in the top right quadrant. As in FIG. 1, the lines connecting the paired points all are extended down to form a complete set of comparative points.

Again, referring back to FIG. 1, there were two reference points, 0 and 13, from which two sets of points were plotted. Through these points, lines were drawn and extended down to form a comparative point, halfway between the two reference points. In FIG. 6, comparative points can be plotted by using the midpoints and only the one set of extended lines. The midway lines are vertical, and these extended lines are drawn at 45° off the x axis, so that the jagged line connecting these intersections comes out at approximately a 45° angle. Since the unit of measure along the x axis is 1, the unit of measure along this 45° line would be √2. The projected point 128, at the end of the jagged line, is 526 units from the zero starting point; this point slides a little to the right of the zero reference point when everything is turned by 45°. Actually, this starting point lines up with √2, the first complex number point. The projected point 128 is 7√2 units up from a 45° line drawn from a point close to the zero starting point. The point 128 is also 56√2 units away from the point where this 45° line crosses the x axis. 56√2/7√2=8. Accordingly, referring again to the point 128, for each √2 unit it moves up, it moves 8√2 units out. The real component of the point 128, then, is 8, or the eighth number out from the start. Thus, its real component is being squared by moving 8 units out. The purpose of FIG. 6 is to show that all the rest of the real components, like the real component 8 of conjugate 128, are being squared, if the same analysis were used on them.

Now, following the example in FIG. 1, the imaginary components of all 64 complex numbers also should be squared and plotted. As before, it is necessary to stretch both the stationary group, centered around the zero reference point, and the moving group, plotted off the conjugate points, up the imaginary axis. Then it is necessary to project their paired numbers to form a new set of 64 common points. This is shown in part in FIG. 7, in which the eight rows of imaginary points in the stationary group are plotted. The right-hand part of FIG. 7 contains a plot of the moving group's imaginary points. As a result, it can be seen for the first time that all these points fall into a very orderly pattern. All 64 points line up diagonally and horizontally, even though their conjugate reference points seem to be random.

In FIG. 7a, the lines connecting the paired points are extended as in FIG. 1. In the first attempt the connecting lines were projected geometrically to the intersection of the halfway points. Because of the enormous lengths of the lines, the actual slopes of FIG. 7a had to be calculated along with the precise intersection points, in order to avoid errors. These calculations are shown in the last number in Column V in FIG. 10. The number in Column V shows the distance along the line back from the intersection point to the y zero axis, plus the distance to the midpoint between the two paired points on that same line. These points are labeled along the entire length of the vertical axis.

On FIG. 7a, the paired grouped points are either left as paired points, numbered as 2s on the left side margin, or are regrouped further into quadruple groupings, numbered as 4s. The quadruple groupings are a matter of relative closeness of numbers, and are not in any way an absolute determination. This closeness varies from 1 up to 12i units of separation for the numbers used, as can be seen in the left side margin of FIG. 7a. The pairing of points 25 with 26, then 34 with 37, then 41 with 45, and 65 with 68, each set very close to the other, seems correct. The close proximity of 34 and 37, as viewed from the vertical axis, separated by only i unit, provided the first clue to the whole idea of the quadruple groups. The separation of the other quadruple numbers was not that clear cut. 10 and 13 were 8i units apart; 58 and 65, 11i units; and 17 and 20, 12i units. 52 and 53, which also were 12i units apart, were not included. The paired points and the other imaginary points in the left side margin of FIG. 7a can be calculated using the following formula. If (a) is the real integer and (b) is the imaginary integer, then the imaginary intersection points are:

$$\frac{- ( {a}^{2} + {b}^{2} ) + ( {a}^{2} + {b}^{2} ) ( 2 - a - b ) + ( 2 \times a \times b )}{2} .$$

The value of the paired points using this formula are shown in FIG. 2, Column VII, running from 2 at 1i to 128 at 868i.

Single point numbers, 2, 8, 18, 32, 50, 72, 98, and 128, which are on the 29° sloping line of FIG. 7, are single points, and have no set of sloping lines intersecting each other to define exactly where they would be on the vertical axis. Although the single points have no set of intersecting sloping lines, they do have an imaginary value. In FIG. 7a, they have been labeled 18 line, 32 line, 50 line, etc., to indicate that they are just a line, and not a precise point. Basically, there are 28 paired points and 8 single points, which will be recombined based on reasons of geometry. The complex number points 2, 5, 8, composed of two single numbers 2 and 8, can be used as a quadruple because the single points can be directed to land right on top of 5 point. The triple point could be made from any single point coupled to a paired point. 32 was chosen as coupled to the paired point 29. The other single points could be moved to be just single points, starting and stopping points, or unknown points.

Going back to the standard general description of the genetic code, it should be noted that the code is derived from different combinations of a grouping of three nucleotide bases, or codons, of the DNA molecule, as transcribed by the RNA molecules to the amino acids. Since there are four possible bases—A, T, C, and G—and these can be arranged in any order or combination, there are 4³, or 64 possible answers. Various combinations of these letters have been associated with different amino acids which are the building blocks of life systems. This association between the amino acids and the codons has been derived experimentally. The codons used in the genetic code are the same as those used in the DNA molecule (except that the outside RNA molecules convert the T base to a U, for Uracil).

FIG. 8 shows the genetic code of the messenger RNA molecule, listing the 64 letter combinations that are grouped to form the 26 groups of codons matched to amino acids. Most of the amino acids are made up of 2 and 4 groups of letter combinations, but two have a one group and one has a three group of letter combinations. The Leucine, Serine, and Arginine amino acids also include both a two and a four group of letter combinations. Three letter combinations are used as starts or stops. As noted before, the DNA molecule transcribes its code to a transfer-RNA molecule which passes the code on to the messenger-RNA. With the aid of the ribosome molecule, the messenger-RNA attaches to the amino acid grouping, thus reading the code of the DNA molecule.

In FIG. 8, the codons are listed first, followed by the abbreviation of the amino acid and then in the aisle, by the number of codons that are used to form each amino acid. The aisle numbers were added by the inventor, but the genetic code itself is well known. The groups of FIG. 2 are derived, as noted, from the converging line placements along the imaginary axis of a mathematical model system. These groups of the complex numbers from FIG. 2 match the groups of codons from the genetic code in FIG. 8. Each has five single items, 12 paired items, one triple item, and eight quadruple items. One group uses a geometric analysis of certain complex numbers; the other, the known experimental analysis of amino acids in the genetic code.

As an example, from FIG. 8 take the first amino acid Phe with two codons UUU and UUC, and then from FIG. 2, the first paired group made up of complex number multiple 53. If this were written as an entire amino acid to which a complex number multiple has been assigned, then phe=53. If the parts are equated, then the first codon UUU equals 2-7i, written as 53-2, and the second codon UUC equals 7-2i, or 53 written as 53-7. This is shown in the first line of FIG. 9 with UUU=53-2 and UUC=53-7. FIG. 9 is a sample codon-amino acid, complex number conversion chart.

The chemical growth pattern of the common amino acids can be stated simply, but it is a very complex subject. Attached onto the carboxyl and amino group of the acid is the “radical,” R, group which is the distinguishing feature of the acid. This group can be very simple, a hydrogen atom, or can be more complex. It could be said that the more complex acids grow out of, or are changed from, the simpler ones and therefore there is a logical set of groupings or series of amino acids. The grouping of amino acids by function forms eight kinds of radical groupings: Aliphatic-nonpolar, Alcoholic-aliphatic-aromatic, Aromatic, Carboxylic, Amine bases, Sulfur bases, Amides, and Imine.

In the chapter on “Information Transfer,” in the book Understanding Genetics by Norman V. Rothwell, there is a series of diagrams which shows the R grouping of the 20 amino acids. These group labels are transferred onto FIG. 2 under the column “Function grouping”. The first group, Aliphatic type amino acids, consists of Glysine, Alanine, Valine, Leucine, Isoleucine, and Serine. Looking at FIG. 8, it can be seen that Glycine, gly, is a four letter combination, GGU, GGC, GGA, GGG. Alanine, ala, is a four letter combination; Valine, val, also is a four letter combination; and so on. By referring to the diagrams in the Rothwell text, it can be seen that the Aliphatic group consists of four fours, one two, and one three, as noted on that chart. From FIG. 8, the rest of the letter combination numbers are transferred over to this chart. This number grouping then is compared to the complex conjugate number grouping of FIG. 2, Columns IV and VI. The two number groupings can be made to match very closely, with one set of 2s out of place.

Once complex number conjugates have been assigned to amino acid function groupings, the process of putting complex numbers to individual amino acids has begun. Based on the amino acid functional groupings, and the residue hydrophobicity, FIG. 2 shows the actual amino acids assigned to conjugate numbers. Obviously, within the group the various four groups could be switched around. Valine, val, could be 10-13 and not 17-20 etc. Glycine, within the first group, is matched to the 2-5-8 conjugate. Glycine is also chemically the simplest of the amino acids, with only a hydrogen atom in the R group position. The acids also may be ordered based on their complexity, but the chart in the Rothwell text seems to have also done that to a certain degree. Thus it can be seen that there is flexibility within the proposed amino acid-conjugate numbers link of FIG. 2. Nonetheless, now there are two possible matches between complex numbers and the genetic code. First came the letter combinations, the 12 twos, the eight fours, the five ones, and the one three, all matching, and now there is a matching of the functional characteristics. One match might have been a fluke, but two such matches makes the possibility of sheer chance less probable.

Once the individual amino acids have been linked to the complex number conjugates, the letter combinations of the amino acids can be assigned to the number combinations of the complex number conjugates. FIG. 9, labeled “sample codon-complex number conversion,” shows this matching. Using the same order from FIG. 8, the first amino acid, phenylalanine, phe, is shown with its letter combinations UUU=53-2 and UUC=53-7. FIG. 7 shows that point 2+7i (53-2) is closer in to the zero x axis than the point 7+2i (53-7); therefore it is logical to list it first. However, this could have been shown as UUU=53-7 and UUC=53-2; that is why the diagram is called a sample matching. The rest of the letter combinations are taken in the order shown on the genetic code and matched to the logical number order of FIG. 7.

Now, let each amino acid occupy one codon spot on the DNA molecular ladder. Pick one letter combination to occupy those three positions. However, from FIGS. 2 and 9, the first amino acid, phe, shows us there are two possibilities for that one spot, either UUU or UUC. It then becomes necessary to determine which one to pick. It should be noted first that the first two letters, UU, in both cases are the same. From genetics, what is known as the “wobble” theory suggests that only 32 sequence possibilities and not 64 may be all that is necessary for the code. (There are four possible nucleotides, in three possible sequence positions, or a total of 64 code variations.) This theory is based on the idea that the last letter of the codon is very weak, so the number of possibilities can be halved. In the case above the last U or C is very weak so there is really only one alternative for that, phe, spot. This would suggest that in the numbering system according to the invention, either 53-2 (2+7i) or 53-7 (7+2i) could occupy the one spot and are almost equal. In the complex conjugate numbering system this is of course the case. Most of the numbers are paired, and therefore there are 36 instead of 32 possibilities shown in FIG. 2 before the final regrouping to 20 amino acids. Thus, like the “wobble” letter theory, the present theory can also be justified. The last letter, as stated earlier, has its own special significance.

Using the 36 possible numbers provides information in addition to the ability to cut the number of combinations in half. In each one of the paired numbers, the real and imaginary components can be interchanged. In the above example, conjugate 53 was the pair made up of 2+7i and 7+2i, so the 2 and 7 numbers were interchangeable. They are interchangeable, but they are not equal, any more than the last U and C of the letter codes UUU and UUC are equal.

Finally, as mentioned earlier, some of the uncoupled numbers 2, 18, 32, 50, and 128, being unused, have no shared positions. The uncoupled numbers seem to have more freedom to move around, and could be used to fill the five unoccupied spots. The uncoupled numbers have no cross comparison lines in FIG. 7a to pin them down, and depend on setting x=y in the formula to suggest where they may be. Also, only two of the single letter combination amino acids in the genetic code are used as amino acids; the others have stop, or unknown functions.

Going back to the code, however, the tying of the letter genetic code to the complex numbers code still may seem incorrect because the genetic code of FIG. 8 seems so uniform, while the complex numbers of FIG. 7a seem vague in some places. First, FIG. 8 is very deceptive. Some of the amino acids barely exist in nature, while others are very prevalent. The gradation of use can range from as much as 0.8 to 100 times. Second, some of the letter combinations on the widely used amino acids are very common, while others can barely be found. The amino acid Proline, Pro, is quite common and therefore the letter combination CCC occurs very frequently, but the letter combination CCA of that same acid is rare. So, if the individual letters of the codons were numbered based on their known existence in nature, they too would be numbered very irregularly.

In assigning the individual codon letter combination to the conjugate numbers, more information can be provided by comparing the codon frequency to conjugate number positions. It is assumed here that if a conjugate number is positioned accurately, then it is more likely to be matched with a codon letter combination that occurs frequently in nature. From the ratings shown in Kreuger, Introduction to Microbiology, p. 327, Table 11-2, the frequency of use of the amino acids can be seen. The frequency of each codon is shown at 100, 20, 4, 0.8, along with the amino acid incorporation percentage. Adjustments from 0 down to 9, are rated 2, and from 9 and below are rated 0.4. These ratings are meant to parallel the frequency rating of the genetic code amino acids. These ratings are one of the many factors that are used in accordance with the present invention to assign specific amino acids to specific conjugate numbers.

Third, FIG. 7a was formed by FIG. 7 which, as has been seen, is very systematic and orderly. Consequently, the remoteness of some of the complex numbers of FIG. 7a, when combined with FIG. 7, may reflect the unevenness of the codon combinations of the amino acids. Finally, when the code was first solved, it was thought that every codon was directly tied to the code. Since then it has been determined that some of the nucleotides are “junk,” and do not relate directly to the code. The irregularities of these codons then also must be factored into any comprehensive theory.

Each integer and conjugate in FIG. 7 relates to a real position on the DNA ladder. Somehow the numbers of FIGS. 7 and 7a are taken back through the RNA molecules to the DNA molecule. This is done with the aid of a common line onto which the general complex conjugate numbers fall. This line is the center fold line along the single point complex conjugate multiple numbers 2, 8, 18, 32, 50, 72, 98, and 128, and is shown diagonally down the middle of FIG. 7 in a solid double line. This center fold line is the key to tying the complex number conjugates to the DNA molecule. In the DNA molecule, the vertical spacing between the steps is generally around 3.4 Å, with 10 turns or 34 Å in vertical height necessary to complete one full turn of the helix.

The diameter of the DNA helix cylinder can be found using the 6.12 Å actual horizontal distance between the steps of the DNA molecule. Then, (6.12 Å×10)/3.14159265=19.48057 Å, the diameter of the helix cylinder. If the curve segment of the helix were flattened between each step, then this segment would be the hypotenuse of a triangle with sides of length 6.12 Å and 3.4 Å, respectively. The horizontal distance squared, 6.12A² plus the vertical rise squared, 3.4 Å, equals 49.0144 Å, the square of the sloping line. Consequently, the length of the side chain between steps of the helix is 7.0 Å+. FIG. 7 is the format from which all the numbers for the code work are derived, and it relates directly to the DNA molecule.

To show the general relationship between FIG. 7 and the DNA molecule, take a piece of paper and draw a 1.8 unit over and 1 unit up sloping line on it, the same slope as calculated for the sloping line in FIG. 7. Roll up the piece of paper into a tube, and this line will make a helix. If FIG. 7 is rolled up into a tube, assuming the horizontal is kept level, it has absolutely no effect on the code placement or the position of points along the fold line. If FIG. 7 is rolled into a very tight roll with many loops of the helix, the distance to get to every point, traveling along the sloping line, remains unchanged. All the points still remain the same height off the ground line of FIG. 7, and the pitch remains 1.8 to 1.

Table A, Quadrants III and IV show the genetic code written in a triangular arrangement which is different from the standard rectangular format of FIG. 8. Quadrant IV shows the fist two letter combinations in the correct relationship to the known amino acid-letter combinations of the genetic code. In row 1, across the top above the double letter combinations is the list of the last letters needed to complete the two letters to form the three letter combinations that match the amino acids in Quadrant III. Bringing these letters down and writing them behind the first two letters completes the codon letter combinations. Then, bringing the letters down from row 2, behind the same first two letters, forms different letter combinations. Now all 64 letter combinations match the genetic code. Take all the amino acids and their letter combinations and rearrange them in the standard format as shown in Table 8. That standard format teaches us nothing. The format of Quadrants III and IV, Table A, shows that the third letter in quadrant IV is being used to reinforce the selection of the first two letter combinations. The grouping of the rows, each having the same last letter, indicates a definite order in the arrangement of the amino acids. The geometry of letter combinations shown in Quadrant IV is logical and shows that the last letter is an organizing factor derived from the particular format used. Put in general terms, the overall format, the picture of the whole, is set, and that determines the choice of the individual parts.

When one accepts that the geometry of Table A is logical with its interlocking parts, then one accepts the fact that there can be a mathematic back-up for the genetic code. It is not a fluke that, if the numbers in the rows of FIG. 7 are slid in the same order to the right side, the resulting format of numbers matches the Table A format of the genetic code. The permutations of 64 combinations derived from two separate geometries being put together are enormous. Consequently, it could be said that it is mathematically impossible that the combinations of these geometries is a fluke. Move up to Quadrants I and II of Table A which shows the integer numbers in Quadrant I that make up the conjugate numbers in Quadrant II. (The third number listed in each box will be explained later.) From Quadrant I it can be clearly seen that the sets of integers are in a logical pattern. The numbers in boxes cannot be moved around. The parts are locked into the format of the whole in Quadrant I, II, III, and IV.

Contained herein is a presentation of a system of numbers based on Gauss's Formula which can be adapted to the new genetic code format. From that section, it can be seen that the Gauss Formula uses a first to last number counting system to calculate the sum of all the numbers in a uniform arithmetic series from the first number to the last number. The sum is equal to the first number “a”+the last number “L”, times the number of terms, n, divided by 2 or (a+L)(n)/2. If the series counts in ones, say 1, 2, 3, 4, 5, L and n are the same number, so the sum equals (a+n)(n)/2=(1+5)(5)/2=15. When Gauss derived the formula, he saw that any arithmetic series could be written as two series, one in reverse order to the other. This is similar to what the DNA molecule is doing. Using Gauss's Formula, a=2 and L=29 (this time counting in 3s) could be written as two series, one in reverse order:

. . . forward 2+5+8+11+29

. . . reverse 29+26+23+20+2

. . . adding the series 31+31+31+31+31

In FIG. 7, two integers x and y were combined to form conjugate numbers. The same two integers x and y can also be combined to form a counting system. In both cases the general format will be the same. The integers x and y in (x+yi)(x−yi) as real and imaginary numbers cannot be added. The integers taken out of the formula as real numbers can be added. If x for the first number and y for the second number integers on the top row of Quadrant I, Table A, were added, the row would appear as 9, 10, 11, 12, 13, 14, 15, and 16. If the integers were −x for the first number and −y for the third number and were added, the products would be the same but have minus signs. All these x and y addition numbers can be used in a summation formula.

The first term plus the last term always equals 31, times the number of terms 10, divided by 2=155. [The number of terms is equal to L+a−1)/spacing between numbers or (29+2−1)/3=10.] It is very important to understand that all of Gauss's summation numbers are applied to forming 64 numbers on one chain.

Table C shows all the conjugate numbers of Table A, Quadrant II, in the same relative positions. Starting on the right side of Table C there is a column with 36 blocks. The top eight blocks are designated with the conjugate number 128. Each conjugate has a certain number of blocks, and these were stacked on top of each other; however, the diagram should have been done with the groups of blocks separated. On conjugate 128, if one were to count up 8 solid segments on the left side and 8 solid segments on the right side, the total would be 16. The L of the Gauss series would be 16, the starting number, a=1 and the number of terms, n=16, so (1+16)(16)/2=136. This number, the summation number, is noted on the opposite side of the number 128, 8 edges away from the 128 as shown by the interior diagonal line. On Table A, Quadrant I, moving on the top row to the left, in the next column the first two numbers are 8+7, which total 15. This would be L and n equal 15, (1+15)(15)/2=120, this summation again shown on the right side of the 113 set of blocks. Following these two examples, the rest of the Table C can be completed.

The summation numbers can be taken from Table C and compared with the conjugate numbers shown in Table A, Quadrant II. Each amino acid has its own conjugate number and summation number. In the top left hand corner of Table C, the conjugate 128 is represented by a set of 8 blocks with 8 solid segments on the left side and 8 solid segments on the right side, thus x=8, y=8. The next conjugate, 113, is represented by 8 solid segments on the left side and 7 solid segments on the right side, thus x=8, y=7. Every single conjugate and amino acid has its own set of blocks with x and y solid segments on each side. Take Table C, flip it over, and shine a light through it for the remaining 28 sets of blocks and amino acids not shown. This right hand version has the openings on the right side.

On Table C, looking at the GLN amino acid position, conjugate 113, from the bottom there are 8 solid sections up to the 113 position, then 7 solid sections down to the 120 position. With the complex number (x+yi)(x−yi), x=8, y=7. Table A, Quadrant I and IV in that position show 8+7−7 in Quadrant I and CAG in Quadrant IV. The 8 is for the first letter C, the 7 is for the second letter A, and the −7 is for the third letter G. Table 9 showed that each conjugate number had two alternatives, in the above case, 8+7i and 7+8i, Therefore, the second alternative for the amino acid GLN with letters CAA, must be 7+8−7. So for the codon CAA, the first letter C would be 7, the second letter A would be 8, and the third letter A would be −8. The first two numbers in Quadrant I of Table C would have to be switched for the 28 codons not shown, and the last letter would be the same as the second letter, but with a minus sign. The top row of this flipped table would read 1+8−8, 2+8−8, 3+8−8, etc. With the addition of these 28 number combinations there are the 64 number combinations required in the code.

Make an amino acid selection and, based on this selection pick, from Table C find the appropriate set of blocks representing the conjugate number that matches this amino acid choice. Each set of blocks is shown with a certain number of left and right solid segments. Count the number of solid segments on the left of the block and use that number on the front line of the DNA chain. This number of solid segments represents the x value. From the number of solid segments on the right side of the set blocks place the number on the middle line of the DNA chain. This number represents the y value of the selection. The above example also works with 28 flipped positions not shown on Table C. The solid numbers would be shown on the front line representing x and rear line representing=y. The front, middle, and rear lines of the chain, using two lines at a time, can house the 8 segment long waves positioned to represent each of the 64 letter combinations of genetic code. Using FIG. 7 and Table C, there is a complete system of identifying each amino acid with a set of units whose maximum number of units cannot exceed eight. From Table C, one can pick a double wave message from the 7 unit long set of blocks with 7 solid segments on the left side and with 4 solid segments on the right side (3 solid segments shown missing on the right) and know that it is reading conjugate 65, the ARG amino acid, codon CGC. Similarly, a 5 unit long set of blocks, thus 5 solid segments on the left and 1 on the right, indicates the LEU amino acid, codon CUU. This system moves numbers over the length of the DNA molecule and to the RNA molecule pushing against it.

The hydrogen bridge of the DNA molecule is governed by the laws of chemical bonding and quantum mechanical bonding. It is known that a cycloid path simulates the motion of this ionized electron traveling between the two loosely bonded atoms on each side of this bridge. A cycloid path is the geometric shape formed by a point on a circle's circumference when that point starts on the ground, rises up as the circle rolls, and then returns to the ground. In this portion of the detailed description of a preferred embodiment of the invention, in order to simplify the text and diagrams, the DNA molecule's length is always shown with its long side in a vertical position. The hydrogen bridge is perpendicular to the long dimension, and therefore the ground line of the bridge is shown horizontally. Also, because this discussion deals with a number of circular motions, this first cycloid circle is referred to as a wheel.

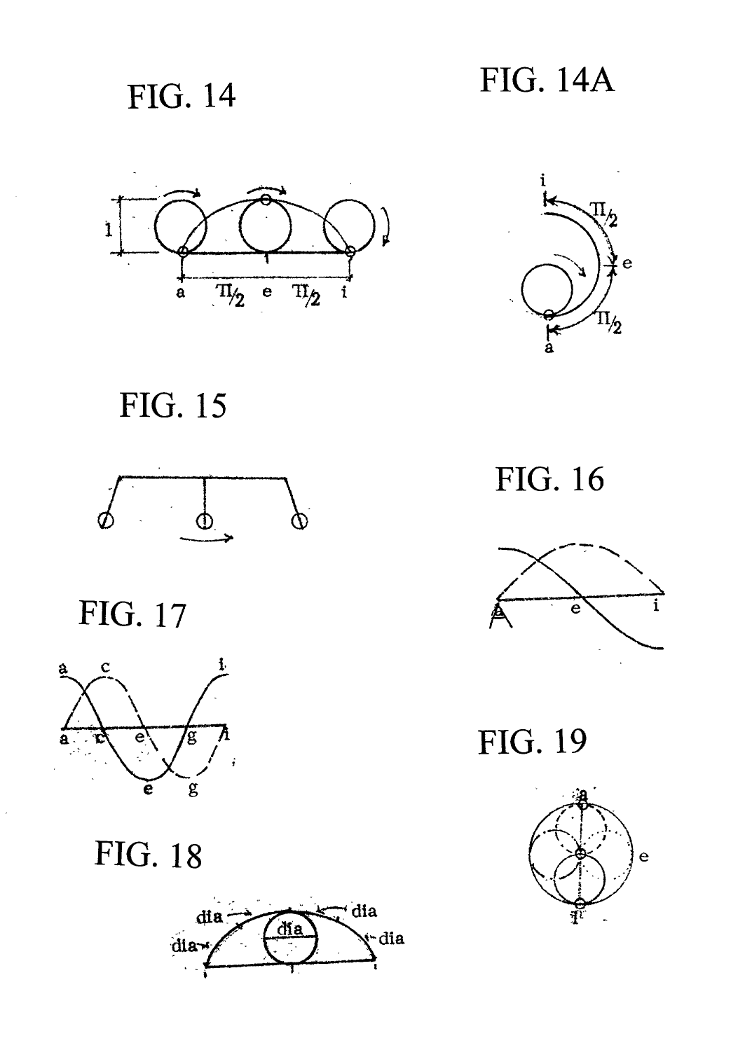

FIG. 14 shows a point on a wheel rising up from the horizontal ground line, rolling across to the other side, thus tracing a cycloid path before returning to the ground line again. While this is happening, the circumference of the wheel comes in contact with the ground line, point for point. If the wheel has a unit diameter, the circumference of the wheel is π, “3.1415926” and the length of the ground line is π. If the length of the ground line is less, the wheel diameter ground line ratio stays the same, but both wheel diameter and ground line size are reduced. If the circumference of the wheel is divided into divisions, then the ground line would be divided into the same number of divisions as the circumference of the wheel rolls on it. In FIG. 14 the circumference and the ground line are divided into two divisions. FIG. 14a shows the ground line bent around in a circle which has twice the diameter of the smaller circle.

When the hydrogen bridge is shown as having a cycloid path formed by a point on a wheel which is rolling back and forth, the wheel is operating as an independent harmonic oscillator. An independent harmonic oscillator also can be represented by a pendulum swinging back and forth, as shown in FIG. 15, again shown horizontally for consistency. Unlike the point on the wheel where the entire system moves from one side to the other, a pendulum is held at a single point, the system being stationary while the bob swings back and forth. However, FIG. 15 shows the different positions of the bob spread out as if the holding point were moving. This modification is provided is for clarity, and so that it will line up with FIG. 14 above it.

Visualizing the motion of a pendulum makes it possible to understand the motion cosine-sine curves of FIG. 16. The cosine curve corresponds to the position and the sine curve to the velocity of the pendulum as it swings. The cosine-sine curves also line up with the wheel's motion forming the cycloid path. As they start on the left, the bob of the pendulum and the point on the wheel are poised to swing and start moving, the position being maximum and the velocity being zero. At stage “a” in FIG. 16, the cosine curve (shown as a solid line) is its maximum positive distance from the center line and the sine curve is on the center line at zero velocity. At stage “c”, the situation reverses, and the velocity of the sine curve is at its maximum and the position of the cosine curve is at zero on the center line. It is easy to see that at stage “e”, the point on the wheel and the bob of the pendulum are going as fast as they can. At stage “e”, the situation reverses again back as it was in the beginning, only the position of the cosine curve is now maximum negative. The cosine-sine curves have completed only a half cycle and continue on another half cycle to return to their starting positions.

This means that the wheel and its point must also continue on another half cycle to correspond to the complete cosine-sine curves. Thus, a complete cycle for the point on the wheel representing the motion of the hydrogen bridge electron's motion would be over and back. To simplify this description, it was thought better if the electron was at one side or the other. Thus, although FIG. 16 appears to be logical as a cosine-sine curve representing the hydrogen bridge, it does not work so well and in fact in the quantum mechanical world it turns out to be quite different.

FIG. 17 shows the cosine-sine curves which have twice the frequency, the amount of up and down motion, as shown in FIG. 16. FIG. 17 is shown on top of and lined up with FIGS. 14 and 15, so this change is easy to see. The sine curve is also drawn very lightly again. This time the curves move through one complete cycle over the length of the hydrogen bridge as the electron moves from stages “a” through “e”. This helps with the quantum mechanical concerns, but it is easy to see that it does not work with FIG. 14. It is known from quantum probability as opposed to classical probability, however, that this will be the case.

Going back to the beginning and the two kinds of waves, earlier the position or cosine curve relating to the cycloid curve was shown. To see where the sine curves were during this process, it should be noted that, referring back to FIG. 17, it is easy to see that the positive cosine and positive sine curves have the same shape but they are out of synchronization with each other. Taking the positive cosine curve, the solid line one, and pushing it to the right by one quarter cycle or 90°, leaving the positive sine curve, the dotted one fixed, the sine curve would be covered completely. The sine curve is shown lightly in FIG. 17, with the cosine curve and positive sine curve, and is the negative sine curve. It, too, is positioned so that it moves in an up and down manner exactly opposite to that of its reflection, the positive sine curve.

FIG. 18 lines up with and shows the identical cycloid path of FIG. 14. FIG. 19 is the same as FIG. 18, but with the complete circle shown. The purpose of FIGS. 18 and 19 is only to show a different way to think about, and draw, the same curve of FIG. 14. The same wheel of FIG. 14 is shown resting on the bottom of a large circle with twice the diameter of the wheel. In accordance with the Cardan Principle, if a wheel rolls around within a circle of twice its diameter, it always moves in a straight line along the diameter of the big circle. Point “a” of the wheel and the drawing point rest on the bottom at the start of the cycloid line. At point “c” the large circle has rolled over to the middle of the ground line and the wheel attached to the side circumference has risen up 90°, with the drawing point still on the circumference of the large circle. Now the wheel is released and allowed to roll back counterclockwise to the bottom again. Then, according to the Cardan Principle, the drawing point will move horizontally across to the center point and back onto the cycloid path line. It is like breaking the cycloid curve into a roll out curved motion with a straight line correction motion. If the big circle is rolled to point “e”, and then the wheel is released again, the point drops vertically down to the cycloid line again. Each time when the big circle rolls and the drawing point leaves the cycloid line, the wheel has to roll back to correct this to return to the cycloid line. Conceptually the base ground line can be divided into an infinite number of segments, the large and small circles rolled an infinite number of times to match these points, yielding the same perfect cycloid line. This drawing exercise breaks the forming of a curve into small increments that can be related to other curves.

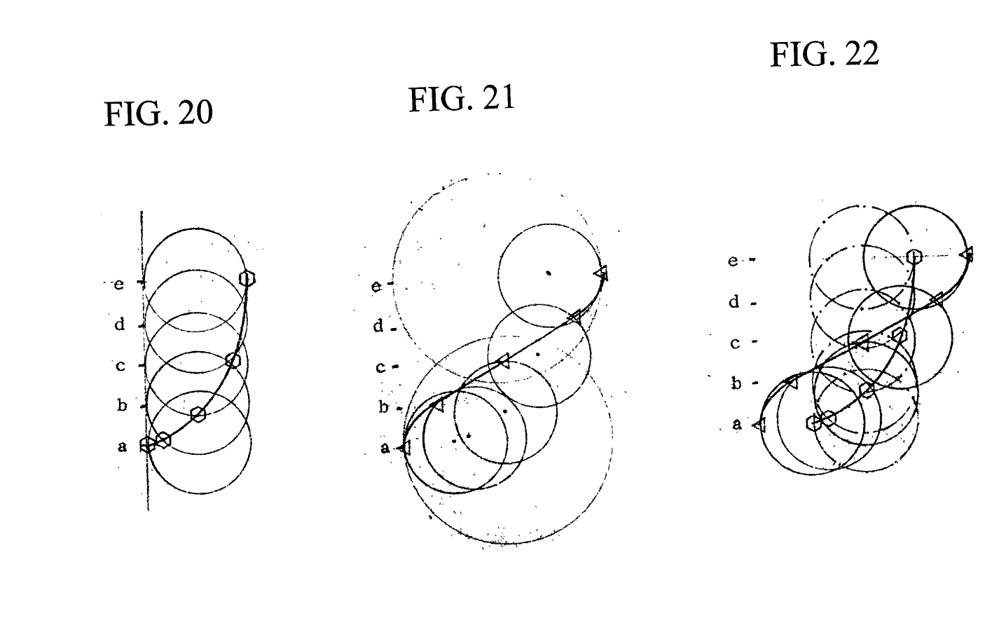

FIG. 20 is half of FIG. 14. With the ground line drawn vertically and labeled starting at the bottom, the four divisions have been marked with the five points “a”, “a1”, “b”, “b1”, and “c”. Tangent to these five points, five cycloid wheels have been drawn along with the corresponding cycloid curve. Where the wheels intersect the cycloid curve, the points have been highlighted with small hexagons drawn around the exact dots. FIG. 21, which is lined up exactly with FIG. 20, corresponds to half of FIG. 17 with only the cosine curve shown. The same set of ground line points “a” through “c” as in FIG. 20 have been shown, but they are shown along a parallel line at the bottom of the cosine curve, to keep the middle of the drawing clear. Projected up from these points to the cosine curve are a set of straight lines. The points where they intersect the cosine curve are highlighted with little triangles. Finally, the large outside first and last Cardan circles are drawn at points “a” and “c”.

FIG. 21's cosine curve can be formed and/or analyzed by using the Cardan Principle again. The large circle is always dragged along its same tangent point always touching the bottom labeled ground line. At the first step, the large outside Cardan circle is dragged backwards from “a” to “a1,” and the small wheel stays in the same relationship inside the large circle. The wheel then is released and rolls backwards one eighth of the big circle circumference, causing the point to leave the bottom line and move horizontally up to the first triangular highlighted point on the sine curve. The big circle is dragged to the next point, “b”, the inside wheel rolls back a quarter of the large circle, 90°, and the point rises straight up again to the next triangle highlighted point of the cosine curve. This process is repeated to form all the other highlighted points each time the inside wheel has started from the tangent ground line point. In each case when the wheel is in the position where it has reached the highlighted points, its center is marked with a fat dot. This series of fat dots defines a series of points which start to form a cycloid line. In fact, if an infinite number of highlighted points are placed on the cosine curve, the center points of the wheel will form a cycloid.

FIG. 22, which is lined up with FIG. 21, shows both the entire cosine and cycloid curves. FIG. 22 is extraordinary because it shows the exact relationship between two very basic functions, a cosine curve and a cycloid curve. The cosine curve should start from the negative position so that this curve can follow the cycloid curve as it rises up. Stating the relationship of FIG. 22 simply, think of the point on the wheel used to form the cycloid, and onto this point attach another wheel of the same size centered on this point. Onto the circumference of this top wheel, put a pencil lined up at stage “a”. As the original bottom wheel rolls from points “a” through to “e” turning 180° and forming the cycloid, this second top wheel turns in the opposite direction, 180°, point for point, the pencil drawing a negative cosine curve. The negative cosine curve is literally riding on the back of the cycloid to ensure that it relates to it at every instant. To make this even more clear, compare points “a” and “c” of both curves. On the cycloid, the point started on the bottom and turned counterclockwise 180° to the top, and on the cosine curve it started on the bottom and turned clockwise 180° to the top.

Going back to FIG. 21, to convert negative cosine point “a” to point “c,” drag the big circle over to the “c” line and roll the little circle backwards or forwards so the point “a” goes from the bottom to the top in a straight line and becomes the point “c”. Alternatively, using the two attached wheel concept, the little circle center slides from the bottom up to the top along the cycloid line, then turns 180° and point “a” becomes point “c”. If the small cycloid and negative cosine circles are attached, the Cardan Principle explanation no longer is necessary. However, it is necessary to remember that the little circle can turn or roll backwards or forwards to get the same answer.

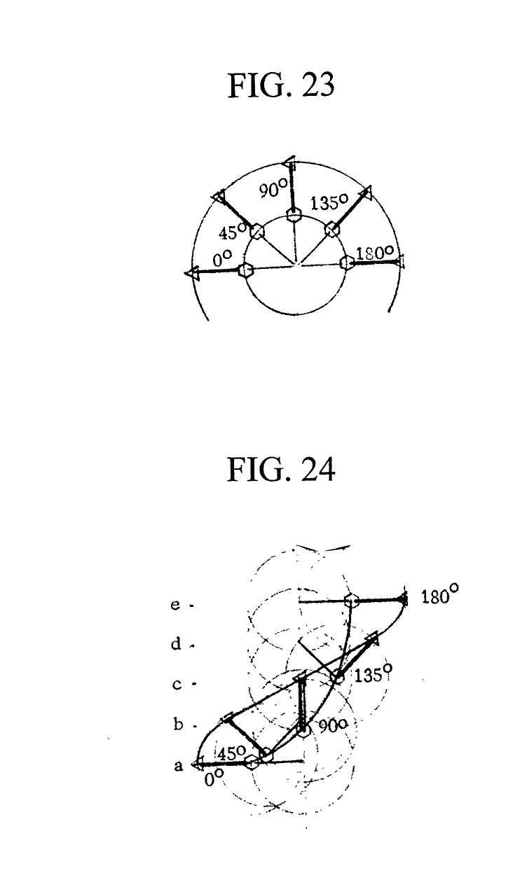

FIG. 23 is lined up with, and is the same as the first half of FIG. 22. From the corresponding points on the cosine curve, lines exactly one-half unit in length (the diameter of the wheel could be one unit) are drawn to the corresponding points on the cycloid curve. Then from these points, again, one-half unit length lines are drawn lightly over to the corresponding points on the center line which is labeled as such. Starting at “a”, there is a half unit length line drawn from the small triangle starting point of the cosine curve to the small hexagon starting point of the cycloid curve. This vertical line then is extended another half unit straight up to center line. Then at point “a-1” a 45° line is drawn from the next small triangle of the cosine curve to the next small hexagon of the cycloid curve. From the small hexagon, the line is extended back on a 45° line to the center line. The next three sets of lines are constructed in the same manner. The one-half unit long lines between the cosine and cycloid curves have turned first 0°, then 45°, 90°, 135°, and 180° off the center line. The combination of the two sets of lines starts straight as the diameter of the wheel, then bends in the middle. By point “b” the two lines coincide and continue to bend through themselves. At point “c” they form the diameter of the wheel again.

If the negative cosine curve points “a” to “c” are projected vertically up to the large circle of FIG. 24, they form a corresponding set of points on half of its circumference. Notice that the backward lettered points are read clockwise. If the cycloid points “a” to “c” are also projected vertically up to the wheel, with the same center as the large circle, they form another set of corresponding points on this wheel's circumference. If the radial lines are drawn, they connect these points, and they then can be lightly drawn continuing to the center of the wheel and the circle. It is easy to see that these radial lines have the same corresponding radial angles as those of FIG. 23. This means that with the angles and one-half unit lengths of radial line points of FIG. 24, one can construct both the cosine and cycloid curves of FIG. 23. One of the easiest things to realize is that FIG. 24 can be moved onto FIG. 23 and position itself so at any point it can be drawing the radial lines that correspond to the changing lines of FIG. 23. All the corrective straight lines of the Cardan Principle can be applied to FIG. 24 so that there are ½ diameter rolling circles as well as the concentric circles.

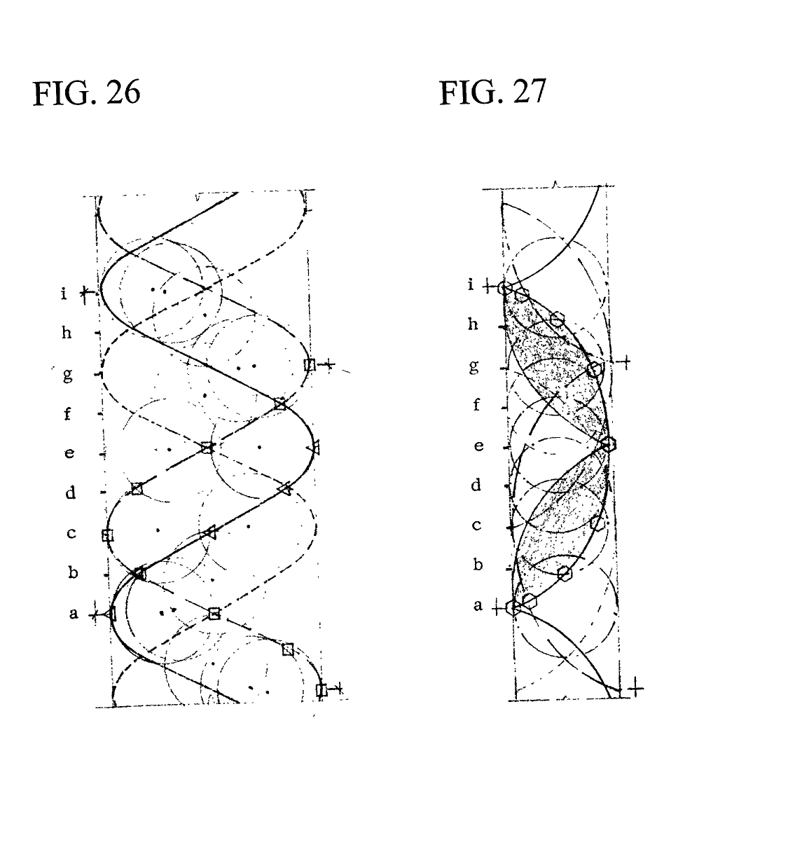

FIG. 26, which lines up with FIG. 21, copies the cosine curve of FIG. 21 and adds the plus and minus sine curves of FIG. 17. The negative cosine curves are omitted. From FIG. 26 it is easy to see the pattern, three curves, then a space, then three curves, etc. Looking at the top of the diagram where the three curves come to the top of their curves, it can be seen that the cosine curve must be in the middle of the positive and negative sine curves, then a space on each side where the not drawn positive cosine curve should be. When the motion starts, the velocity or sine curve is usually at zero and the cosine curve at +1 or −1.

The dual bridges concept was a simple enough idea. The DNA molecule divides in half down the middle between its nucleotides or paired bases. Connecting these bases in the middle is, of course, the hydrogen bridge and a hydrogen atom. When the entire DNA molecule divides, each side, composed of the outside chain and the inside nucleotides, replicates itself and the complementary components are formed. This is done instantaneously as the long molecule is dividing. For this to happen, the new parts must be in exactly the right place at the right time.

The starting point is a cycloid, or many repeating cycloids, as the ionized electron bounces furiously back and forth between the loosely bonded sides. If the cycloid and the hydrogen bridge are cut in half in the middle, the result is half a cycloid and half a bridge cantilevering out from each side. The accepted solution is generally that the electron and therefore the entire bridge ends up on one side. This means that the other side is left with nothing. A different solution might be that, since there are dual spans to the bridge, when the division occurs each side ends up with at least an image of what had been there. This helps both halves of the dividing chain to direct their approaching parts to finding the right place for the new bridges that are being formed.

FIG. 27 shows the cycloid curve in a solid dark line with the small hexagons on it. This is the curve that was used to form the cosine curve. Then, an extraordinary thing happens. There is another cycloid curve that can also form this same cosine curve. This curve springs from an upside-down ground, a second ground line, not on the bottom but on the top of the diagram. This cycloid curve is shown moving along with the original cycloid curve in the same direction, each one of them being formed by its own rolling wheel. The area in between the two alternate cycloids used to form the cosine curve has been shaded. FIG. 26, which is lined up with FIG. 27, shows the common cosine curve to these two cycloid curves. By comparing the two Figures, one can see that the common cosine curve always stays between the two opposite cycloid curves that generate it. This opposite cycloid has its own drawing top wheel, which generates, point for point, the same cosine curve. This is done by having its new top wheels rotate in the opposite direction off its bottom cycloid wheel. This opposite cycloid is 180°, or one-half cycle, out of synchronization, so that its mid-point corresponds to the starting point of its respective opposite curve.

It is these two cycloid curves that can be used to form the dual span concepts of the hydrogen bridges. That would yield not only a means of dividing the hydrogen bridges in half, but also, if FIG. 26 is increased in scale, it would be a means to divide the opposite side chains from each other. Again, each chain, like each span, would be of a dual nature, carrying the dual information necessary for replication. Before leaving FIG. 26, it should be noted that on each side of the main cycloid curves are other cycloid curves used to produce the sine curves. These identical cycloids are all spaced one-quarter cycle apart, as were the sine curves and cosine curve that they generate.

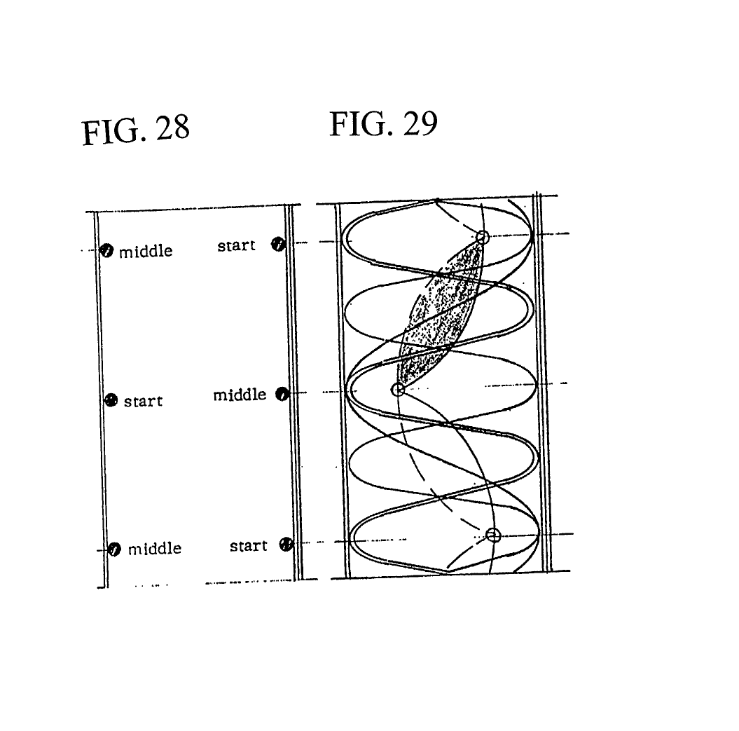

Jumping up to FIG. 28, it can be seen how the dual side chain concept works as it divides the DNA molecule. In FIG. 28, the whole cycloid has been turned upside-down as viewed from the left side, so that its shaded area between the right side up and the upside-down cycloids will line up with the same shade area of FIG. 27. FIG. 28 starts with FIG. 26, and one set of two alternative cycloids. The next step is to take the square of the single wave amplitude shown and square it mathematically. In quantum mechanics, when the wave amplitude is squared, the result is the probability curves and the probability. Holding the sheet sideways with the triple line at the bottom, it can be seen that the amplitude curves swing up and down across the center line, creating negative and positive values. When it is squared it creates only positive value curves as read from the triple line at the bottom. This probability curve is shown with a double line. It should be kept in mind that the cosine curve is measured from the center line, but the probability curve is measured from the bottom triple line. The probability curve is twice as high as the cosine curve at its maximum point. The three high points of this curve are where a particle is most likely to be found. In FIG. 29 on the triple line there are three particles shown with solid circles at these points. These points have been labeled start, middle, and finish to correspond to the start, middle, and finish point of the whole cycloid shown in FIG. 28.

If the sheet is turned around to look at FIG. 28 with the double line at the bottom and the same wave amplitude curve as above, and it is remembered that there is one cosine curve which is tying the two cycloids together, the next step is, with the double line as the ground line, to again square the one curve. This squaring produces another set of probability curves, shown this time with a single line. Next, the three high points are found, and are shown on the double line ground line of FIG. 29 with solid circles. These three points line up with the top three but this time they are labeled middle, start, and middle to correspond to their cycloid. FIGS. 28 and 29 reveal that working with a cosine curve, and looking at it from the triple ground line, when the cycloid is started, there probably will be a particle at the start, one at the mid-point, and one at the end.

When the sheet is turned over, the same thing happens, only this time the points line up but are 180° out of synchronization. Quantum physics thus yields the same results as geometry, thus verifying the concept of upside-down cycloids tying waves together. It also reveals that if this dual theory is applied to the hydrogen bridge, because of its scale, the particles only will be found at these three points as shown on FIG. 10.

The actual distance across the front hydrogen bridge of the A-T nucleotide base is 2.82 A and across the front C-G nucleotide base is 2.84 Å. (page 54, Life's Other Secret by Ian Stewart, John Wiley+Sons). Both are close to 2.8 Å and might have that value depending on a slightly different paracrystal condition. The sloping distance along the side chain between phorphorous atoms as calculated above is close to 7.0 Å (page 317, Molecular Biology by E. J. DuPraw). The factor of 10/4s converts the hydrogen bridge crossing to the single side chain length. FIG. 10 is a diagram showing how the integer numbers and their representative waves are applied to the hydrogen bridge and the side chains. The top group of lines represent the side chains and the bottom group the hydrogen bridge. The important distinction between these two groups is the side chain, representing a codon, which is one continuous line made up of the three ladder steps. The three separate hydrogen bridges, representing a codon, are three separate lines used together. Three combined 7.0 Å long segments of the outer chain of the DNA molecule that form a codon are shown in three lines, the front one for the x wave, the middle one for the +y wave, and the back one for the −y wave. The x wave is divided into 8 parts starting at the beginning of the codon, and extending over two ladder steps, and is made up of the four waves shown with loops. The middle section starts ½ wave length back, and is also divided into 8 parts with 4 waves, the −y waves, starting 1½ wave length back from the start. The positioning of the x, +y, and −y waves is consistent with FIG. 26, and the use of final wave positions is consistent with FIG. 29. The 1 to 8 integers have been shown on all three lines in their correct positions. Note that the total length of the codon is almost used up by the number-wave parts.

The three separate sections of the hydrogen bridge are shown with their respective numbers and waves. The x wave at the top would start counting at the first hydrogen bridge. The +y and −y also counting, beginning at the start but ½ wave back in relation to the front hydrogen bridge. FIG. 29 clearly shows that each hydrogen bridge can only exist and predict positions in three places, at the beginning, in the middle, and at the end. It therefore takes three separate hydrogen bridges working together to make the selection of 1 to 8 positions, thus 1 to 8 integers, not counting 0.

Looking at FIG. 10, each position is separated by a distance of 2.8 Å/2=1.4 Å. The three 2.8 Å wide hydrogen bridges are separated by two 1.4 Å gaps on this diagram. Not counting 0, the 1 to 8 positions on the side chains are separated by 7 Å/4=1.75 Å distances. The 1.4 Å long distances on the hydrogen bridge can be converted to the 1.75 Å long distance on the side chain by multiplying by 10/8. Point 6 on the x wave of the hydrogen bridge of FIG. 10 is 6×1.4 Å=8.4 Å from the starting point of the first hydrogen bridge, allowing 1.4 Å between integers 2 and 3 and 5 and 6. Point 6 on the x wave of the side chain is 6×1.75 Å=10.5 Å from the starting point. With the value of y=4, there would be a point 4×1.4 Å=5.6 Å from the start on the hydrogen bridge and 4×1.75 Å=7.0 Å from the start on the middle chain. From Table C, 6 blocks on the left and 4 blocks on the right is conjugate 52, which from Table A is the PRO amino acid with a letter combination of CCC. Using a selection from FIGS. 7 and 10 and Tables A and C, there is a position for each amino acid selection on the side chain and hydrogen bridge.

The hydrogen bridge waves can be brought over and onto the side chains to join the side chain waves. Then both sets of waves can be used to focus on the x=6, y=4 peak points of the PRO amino acid selection. Again, for the x value, the side chain would be 6×1.75 Å=10.5 Å from the start using 3 wave lengths. The x value for the hydrogen bridge would be 6×1.4 Å=8.4 Å using its total wave lengths going to the side chain. This wave could run 8.4 Å and be 10.5 Å−8.4 Å=2.1 Å short of the end point of the side chain wave. If the hydrogen bridge wave starting point on the side chain is moved forward 2.1 Å, its end point would coincide with the side chain's end for the x=6 selection. FIG. 29 shows that end points are peak points for the waves that are being used. The different wave length waves would run on top of each other and be out of synchronization until the point where their peak points coincide. At this point, 10.5 Å from the start, the two waves reinforce each other, and the x integer selection is made.

On FIG. 10, directly below each of the 1 to 8 integers, are the distances of the 8, 1.75 Å side chain increments and the 8, 1.4 Å hydrogen bridge increments. The difference of the two numbers is noted below each column. The difference of the column goes from 0 to 2.8 Å and shows that the 2.8 Å span of the hydrogen bridge gap is being mimicked over the span of a codon. This occurs because the three hydrogen bridges are working together to select one integer point. Under the integer 6 is the difference value of 2.1 Å, made up of six, 0.35 Å increments. If the 0.35 Å increments of difference in wave length could be brought to the side chains, then an adjustment in the starting position could be made. This would permit the two waves to reinforce each other. These 8, 0.35 Å increments are shown in FIG. 26 labeled a to i. FIG. 26 was used to form FIG. 29, which was used to derive the position points for the hydrogen bridges.

The above paragraph provides the key as to how the genetic code is placed on the DNA molecule. FIG. 10 and FIG. 26 list and show the cosine wave, x, the sine wave, +iy, and the other sine wave, −yi, but missing in both the diagram and figures is the other cosine wave, −x, which straddles the sine waves. It would be 90° off the +yi and −yi sine waves and 180° off the positive cosine wave. When −xi is used in the process (−x+yi)(−x−yi) and multiplied out, it forms the same answer as +x. The three letters of the genetic code can be assigned to the three waves. The first letter would be assigned to the x wave, the second letter to the +iy wave, and the third letter to −iy wave. Remember from Table A, Quadrant I where the three numbers were shown, the second and third numbers were the same but with first a positive sign, then a negative sign. In all cases but one (amino acid Met.), in the genetic code when the first two letters are fixed, changing the last letter does not change the amino acid selected. Changing the last letter changes the variation selected of the same amino acid. Changing from +iy waves to −iy waves makes this selection possible. Three waves, x, +iy, and −iy are mathematically necessary to produce the conjugate numbers of the genetic code. That is the strongest reason found to date why the codons are made up of only three letters.

If the conjugate numbers of FIG. 7 in a holographic process represent the whole picture, then the x and y integers that form the conjugate numbers represent the parts of the whole picture. The x and y integers can be used on the side chains to represent the conjugate numbers. The incredible feature of a holographic process is that the sum of the parts equals the whole, which is normal but in addition the whole picture can be found in each of the parts. This kind of flexibility occurs, not because the conjugate numbers can be found in each of the integers, but because each of the integer waves comes with two parts. The front x wave shown in FIG. 10 on the x wave line is followed by a +yi or −yi wave on that same line. The middle +yi wave is followed by a x wave on that same line. Multiplied out (x+yi)(x−yi), they produce the conjugate number on each of the x wave and +yi wave lines. In addition, as will be shown later, they also produce (x+yi)(x−yi)=1 process on each of the x and +yi, and −yi lines of the hydrogen bridge and side chains.

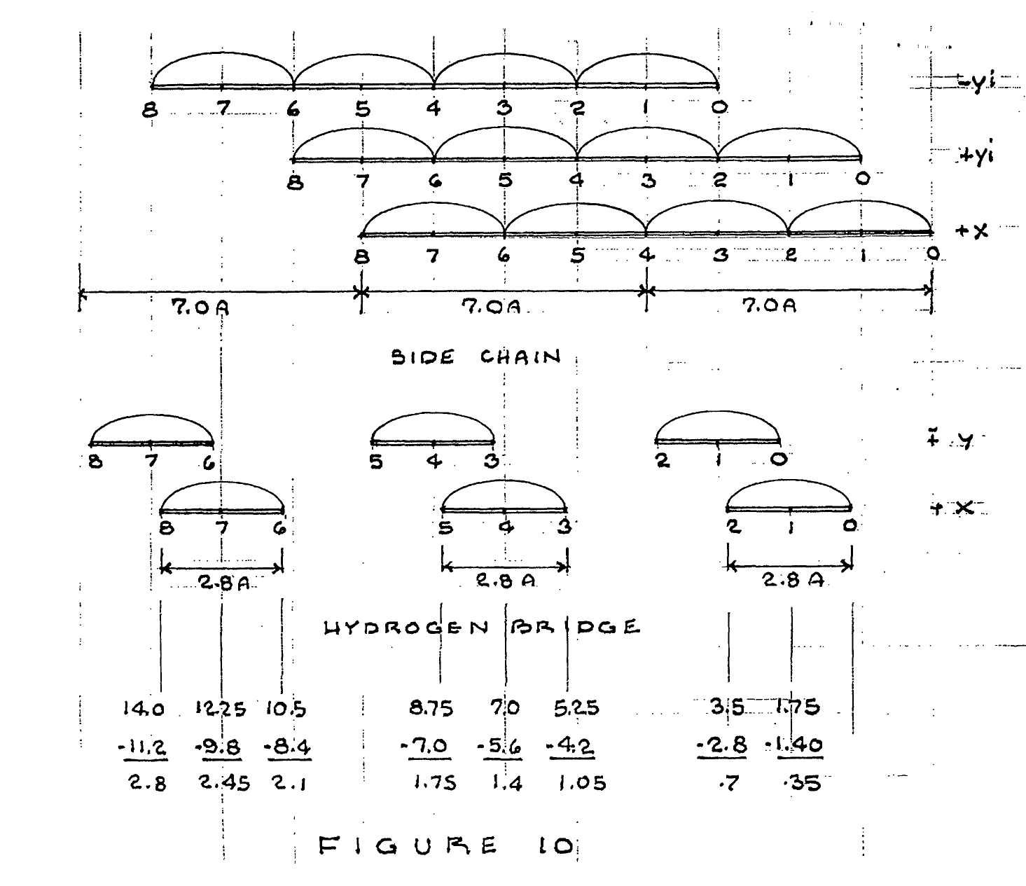

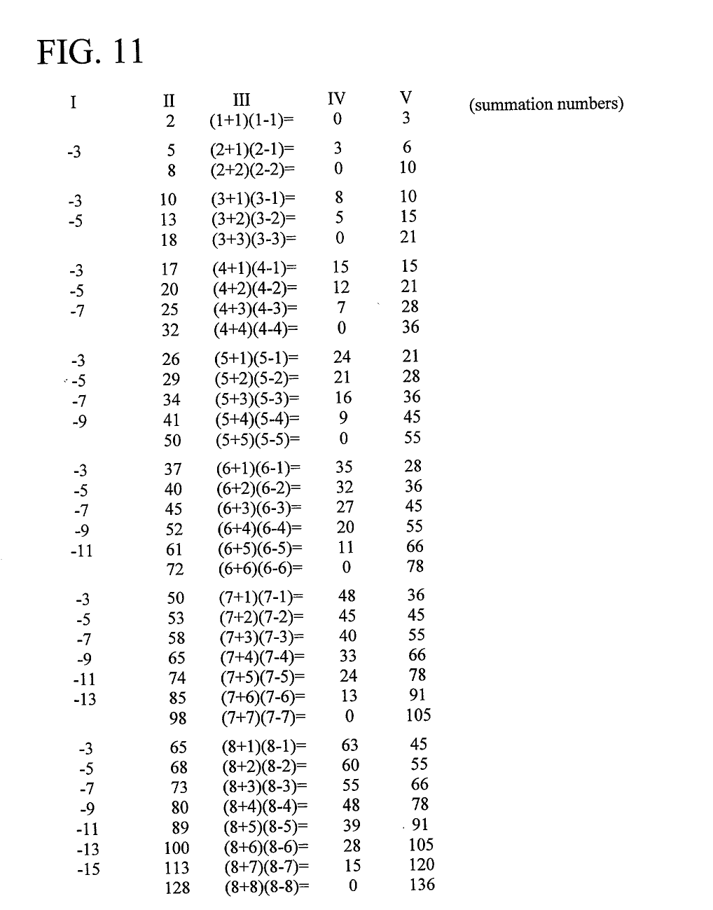

The flexibility of the conjugate numbers derives from the fact that they can be arranged in a very orderly manner. The bottom set of numbers of FIG. 11 shows a set of conjugate numbers in Column II and their respective differences in Column I. These conjugates come from the top horizontal, 64i line of FIG. 7. Column III is the integer presented in the (x+y)(x−y) form and not in the (x+yi)(x−yi) form that derives the conjugate numbers. Used in the (x+y)(x−y) form they result in the real product numbers shown in Column IV. When each of these real product numbers is added to its respective conjugate number, they form the totals shown in Column V. When the real product numbers are subtracted from their respective conjugate numbers, they form the totals shown in Column VI. The next to the bottom set of numbers of FIG. 11 shows the conjugates of the 49i line of FIG. 7. The numbers on the 25i, 16i, 9i, 4i, and 2i line of FIG. 7 are filled on FIG. 11. There are 36 conjugates minus the 8 nonpaired conjugates equaling the 28 that are shown on FIG. 11.

The left side of Table B shows these 28 conjugates in the same format as Table A. On the right side of the vertical center line of the 8 non-paired numbers are the other 28 numbers not shown on Table A, which form the complete 64 conjugate numbers. Below all the conjugates are their respective (x+y)(x−y) real product numbers. In the far left column are the conjugate real product numbers for the left block and along the top right row for the right block. The two sets of addition numbers are perpendicular to each other. The significance of the these addition numbers is that they are the same as the non-paired numbers which are set on the 29° sloping line. That means that the 2 dimensional Argand Plane numbers of FIG. 7 can be transferred to a single 1-dimensional line. This transformation is done by adding the whole picture conjugate numbers to the integer product numbers. Using a holographic process, the conjugate numbers of FIG. 7 can be put on the single line of the DNA molecule. The 64 conjugate numbers have been converted to 8 positions on a single line but they were derived from the Tables A and C format. Table B coupled with the side chain summation wave numbers reinforces that concept of transforming the 2-dimensional FIG. 7 to a 1-dimensional set of numbers.

Visually, one of the strange things about the DNA molecule is the existence of the codons. The codons, as mentioned earlier, are the groupings of three nucleotides in a row used to form the genetic code. In the spacing of the nucleotides, there is no atomic break, no extra space between the bases, rungs of a ladder, nothing, that would indicate that after reading three bases, one starts with a new group. One would at least expect three rungs, a missing rung, then three more, one missing, etc. This is not the case, and it may be the reason that there have been no answers to this puzzle to date except to say that it exists, and therefore it works. Using only +x, +yi, and −yi and not −x, three waves indicate that there is a very strong wave-particle function that causes the codons.

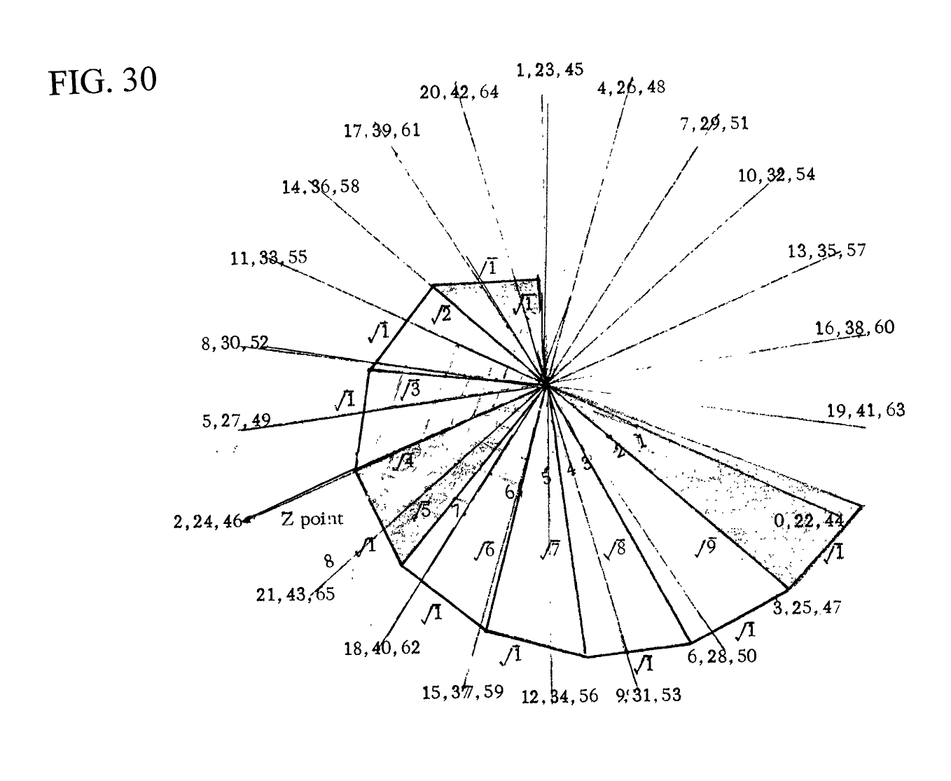

On FIG. 30, the first triangle has sides of √1, √1, and √2, or 1, 1 and √2, remembering that the sum of the squares of two sides equals the square of the hypotenuse. Next, if the √2 becomes one of the sides with the other being √1, the hypotenuse is √3, or √2²+√1²=√3² or 2+1=3, and so on. FIG. 30 shows this old mathematical device built into a spiral where one side of each triangle is √1. If this diagram were extended, it could grow very large. For example, the formula could grow to √2000²+√1²=√2001². This diagram could also be changed to form a −√1 spiral using the same mathematical device. If above the center point are √1, then below the center point there must be −√1. In FIG. 30 the first −√1 right-angled triangle is shown in dotted line form, to show how this spiral would form up. This series of square roots would then be plotted as an opposite-hand spiral.

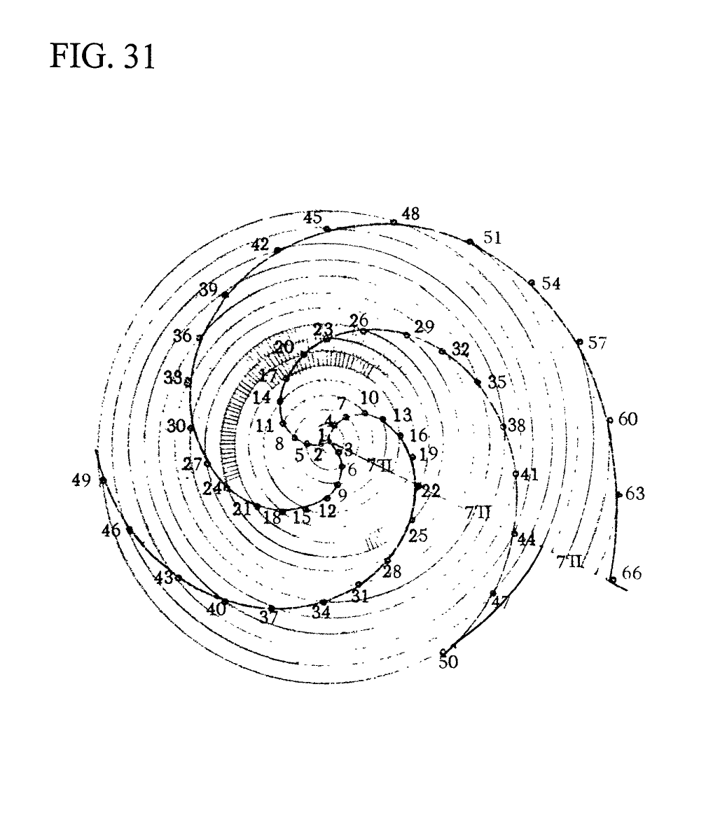

FIG. 31 is the spiral of FIG. 30 continued from the triangles with sides of √1 on out to √4,356 which is 66², and is reduced to about 1/66 the scale of FIG. 30. The spiral line formed by the √1 sides of the triangles has been shown out to 66², 4356, but only a sample of the very outer portion of the triangles' long sides have been indicated, around 24² to 26². At √4,356, there would be a right triangle with one side of 66 units coming from the center, a side of one unit on the spiral, and a hypotenuse of √4357. If the long side and the hypotenuse of all these wedge shaped triangles had been drawn, the middle of the diagram would be black from lines. In addition to the √1 spiral, in FIG. 31, a second set of spirals are shown connecting the square root integers that are separated by 3. Thus 1, 4, 7, 10, 13, 16, 19 etc. and 2, 5, 8, 11, 14, 17, 20, etc. and 0, 3, 6, 9, 12, 15, 18, 21, etc. become the three spirals that group all the integers. Reading these numbers toward the center of the spiral, they become 19, 16, 13, 10, 7, 4, 1; 20, 17, 14, 11, 8, 5, 2; and 21, 18, 15, 12, 9, 6, 3, 0.

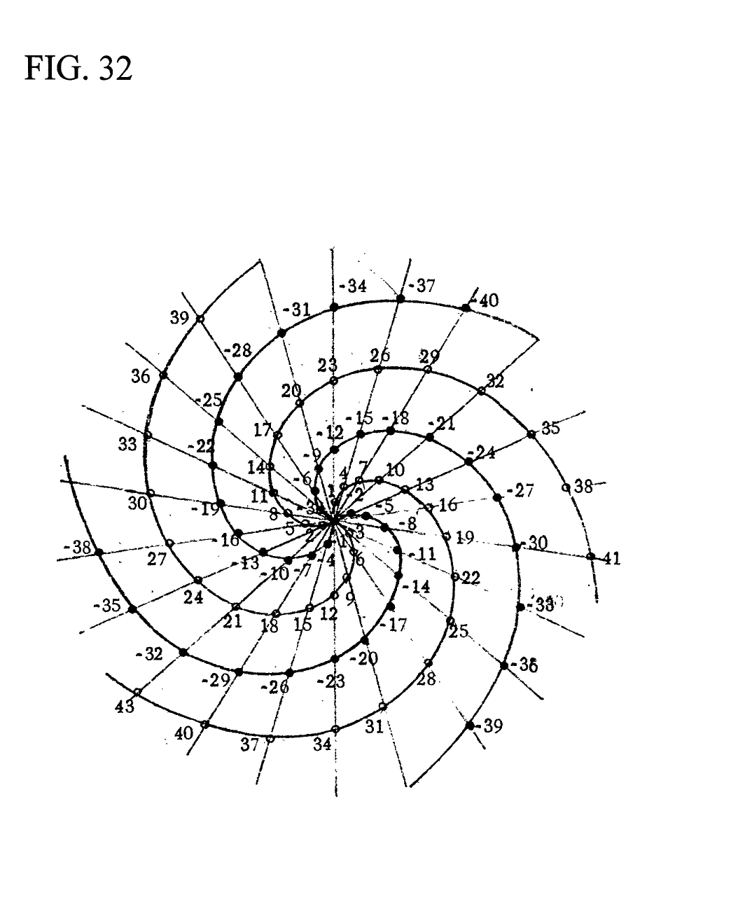

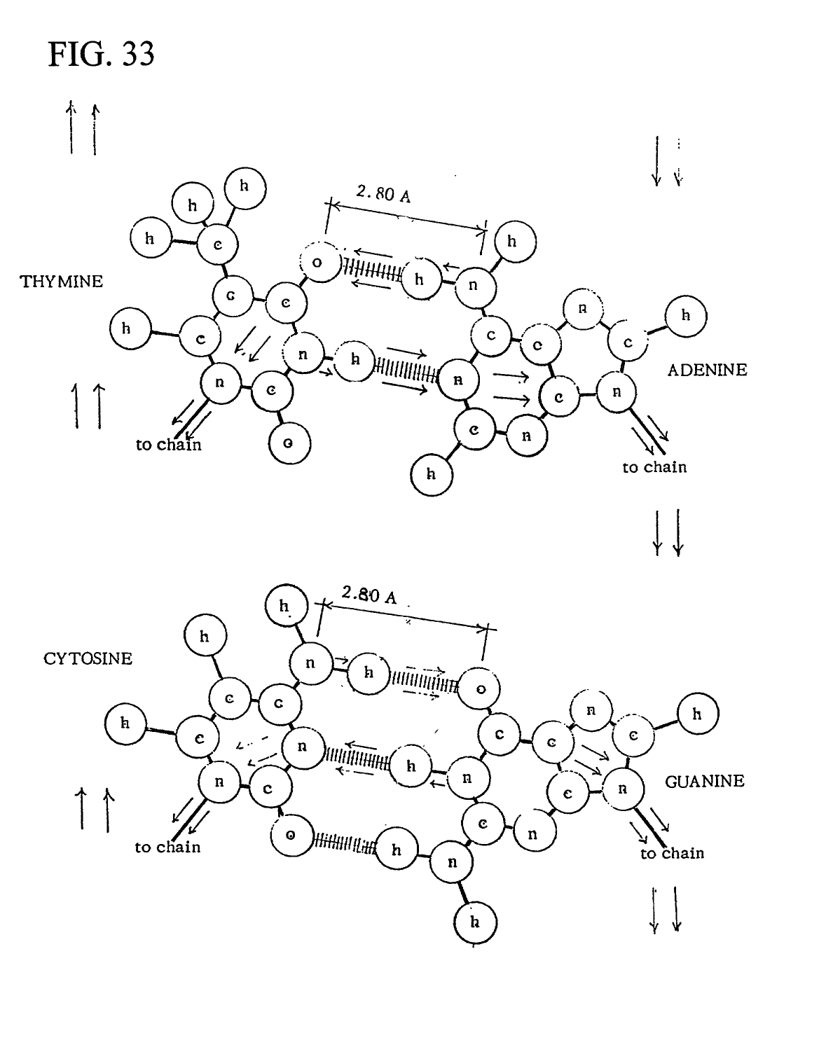

FIGS. 30 to 32 have a system of right triangles where one side is always 1, no matter how big the other leg of the triangle is. Every step counted on the DNA ladder is always geometrically brought back to the starting point. The numbers on the gradual spiral of FIGS. 30 and 31, formed by the square root of −1 triangles, can be applied to the circumference of a circle. In FIG. 31, as the numbers get larger, the distance from the center point to the gradual spiral line becomes greater. When this distance becomes very large, the separation between the overlapping gradual spiral lines becomes small by comparison. The overlapping spiral lines in effect join, and the gradual spiral lines become the circumference of a circle. When this happens, the numbers behave as if they were on the circumference of a circle. On FIG. 31, look at the positions of the numbers 48, 49, 50, and 51 on the square root spiral. Notice that moving around on this gradual spiral's circumference, with theses numbers, a whole circle is almost completed. Point 51 is very close to the starting point 48. On FIG. 31A, in a similar manner, small circle point 3 is very close to the starting line small circle point 0. If small circle points 1 to 3 represent a codon, each codon is almost the complete circumference of a circle. It only needs 1/22 of the circumference to complete the loop, when the circumference has been divided into 22 segments. FIG. 32, which has the numbers placed on 22 radial lines, shows that a codon set of numbers almost completes the loop, no matter how big the numbers get. When the numbers are huge, as they would be on a long DNA molecule, the gradual spiral becomes a circle, so FIG. 31A would become a fair approximation of FIGS. 30 to 32.

In FIG. 31A, when the circle is rolled out onto the straight tangent line passing through the small circle point 0, the circumference numbers fall on the straight line as shown. These numbers (in small circles) represent each step of the DNA ladder and are also shown in small circles on the x wave line of FIG. 10. The numbers, not shown in small circles on the tangent line of FIG. 31A, are also produced by the rolling circle. They also tie FIG. 31A to FIGS. 30 to 32, and their function will be shown later,

In this manner, it is possible to tie the system of FIG. 10 to that of FIGS. 30, 31, and 31A. From these spirals of threes, it can be said that the first step of the codon starts at point 0 and goes to point 1, the second step from point 1 to 2, and the third step from 2 to 3, the beginning number signifying the step number. FIG. 32 shows the three 3s spirals of FIG. 31 at the same scale. Straight out from the center connecting all the 3s integers are radial lines. As noted, the radial lines are equally spaced, and divide the 360° turns into 22 equal segments or arcs of 16.36364 . . . degrees. FIG. 32 also shows the three comparable −√1 3s spirals spaced in between the positive integers. FIG. 30 has the 22 radial lines of FIG. 32 brought back to the center of all the spirals and the beginning of the right triangles. The numbers of FIG. 32 have been carried out to +40 and −40 covering both the positive and negative 3s spirals.

Looking back at FIG. 30, it can be seen that, between each one of the shaded triangles, there are several non-whole integer triangles that are not used in the basic counting system. From √1 to √4 there are two non-whole integer triangles, √2 and √3. From √4 to √9 there are four, √5, √6, √7, √8 from √9 to √16, six, and so on. These groups of non-whole integer triangles are increasing in an arithmetic progression by 2 each time. Of course, only whole integer triangles are used, but there may be a use for these non-whole integer triangles. The number of non-whole integer triangles grows, but the number of radial lines between each new whole integer stays constant. Looking at FIG. 30, it can be seen that there are seven radial pie-shaped segments from 2, (2, 24, 46) to the next whole integer 3 (3, 25, 47). Between 3 and 4 (4, 26, 48), there again are seven, and so on. On the top edge of FIG. 31A, there are always seven constant size divisions between each integer, even as the integer numbers get bigger.

The numbers from FIGS. 30 to 32, and FIG. 31A, which is tied to FIG. 10, form a logical system for counting the DNA ladder steps, and show the numbers grouped in sets of three for the codons. As has been said before, one of the reasons there are codons made of three steps is that the first step represents the x wave, the second step the +yi wave, and the third step the −yi wave. Looking at FIG. 10, however, the possible positions of the x, +yi, and −yi wave lines span over three steps of DNA molecule. So from FIGS. 30 to 32, the steep spiral numbers 1, 4, 7, 10, etc., 2, 5, 8, 11, etc., and 3, 6, 9, 12, etc. can only generally be associated with the x, +yi, and −yi waves. On FIG. 10, the distance between the small circle numbers 0 to 1, 7.0 Å long, is divided into four parts. On FIG. 26, a wave length is divided into four parts using the points at a, c, e, and i. The x, +yi, and −yi waves peak at points a, c, and g. The 0 starting peak points of FIG. 10 are shown in this same relationship as the a, c, and g points of FIG. 26, adjusting for the missing −x wave. Each of the x, +yi, and −yi waves are ¼ wave length from each other. The +yi wave line and the −yi wave line of FIG. 10 can be superimposed onto the x wave line so that they represent separate waves moving along a single line. In a similar manner, using the +yi wave line as the base, the x and −yi wave lines can be superimposed on it. Finally, in the same manner, using the −yi wave line as the base line superimpose the x and +yi wave lines on top of it. These three sets of new wave lines, each holding its own and the others' number information, can be thought of as the multi-dimensional source of the integer numbers.

Each set of waves always has three wave parts, but one of the wave parts has no numbers. Drop the second or third composite wave lines depending on whether the +yi or −yi wave is used. Now there are two composite lines, each made up of three waves in a row. All the selected integer numbers are still used, but they are represented on the composite single line formations, This make up of waves relates directly to the fact that although there are three nucleotide letters in a codon, the selection of amino acids is made basically from the first two. Also, as will be seen later the two sets of composite wave lines form the volume set of fringe lines used in a volume holographic plate. This another way of explaining why the (x+yi)(x−yi) formula is mathematically a description of a series of waves coupled together.

Mobius

Take a long strip of paper and mark it off in an even number of increments, First fold it in half and join the ends. Imagine that the top edge loop represents one DNA molecular chain and the bottom edge loop represents the opposite chain. Waves traveling on the top loop would be completely independent of wave traveling on the bottom loop. It is known that the two chains of the DNA molecule work together, so that this arrangement cannot represent the DNA molecule. Now, disconnect the ends, give the edges a half twist, and rejoin them. Now the formerly separate edges are joined, and the new structure is a Mobius.

FIG. 40 shows the formation of a flattened Mobius. Take that same strip of paper, left end points marked A and B, right end points marked A′ and B′ and fold it along the solid diagonal line C-E′-C′, where E′ is the mid-point of the total strip. The left side will end up lapping the right side as shown in the diagram, but the two edges are still separate. Fold the strip again along the E-E′ dash-dot line so that the original right side still remains on top of the original left side, point C being on top of point C′ and points A and B′ and points B and A′ are joined. The double folded shape will appear as the third diagram on FIG. 40. When the fold lines are separated, it is easy to see the 3-dimensional Mobius figure that has been formed. Flattened again, it is apparent that the angle of the first fold is arbitrary but the line of the second fold is always perpendicular to the first fold. Move the end points A, B′ and B, A′, holding them vertically back to the upper edge of the second fold line marked point E and the point vertically above it marked F. Throw the rectangular portion formed by points A-B′, E, F, and B-A′ away and notice that the shortened flattened Mobius is still a Mobius. The length of the Mobius can be any incremental size and the angle of double fold can be adjusted.

Imagine taking apart and unfolding the Mobius of FIG. 40 to form a flat strip of paper. Imagine this represents the particular conjugate number Mobius chosen from the third set of Mobius. Notice that the first fold line, AB″ to E is part of a right triangle, That triangle is formed by a horizontal length on the top edge, a unit length from the point E to small circle 0, and the above fold line. Looking at FIG. 30, it should be noted that this triangle is identical to the right triangle having the same conjugate unit value. FIGS. 30 to 32 can be spread out on a single line to form a whole series of right triangles matching those in the spiral. Thus FIGS. 30 to 32 can be related to the third conjugate number Mobius in this manner. This comparison requires that the height of the flattened Mobius be set to match the unit distance between each small circle step. The Mobius taken from FIG. 40 uses a strip of paper whose top and bottom edges represents the opposite chains of the DNA molecule. If the height of the strip in FIG. 40 were cut way down, a thinner flattened Mobius could be formed. The horizontal points used would not be changed, and integer-conjugate number functions would not be altered. In other words, the height of the strip is variable, and could represent a changing wave height and amplitude.

FIG. 10 placed the integer numbers on the x, +yi, and −yi wave lines of the DNA side chain. These three separate wave lines were put together on the x wave line starting at small circle 0 and running over the length of a codon to small circle 3. Line up the increment point E′ of FIG. 40 with the small circle 0 increment point of FIG. 10. Starting at this point, mark off the x wave line with its integer spacing over a codon length onto the top edge of the third diagram of FIG. 40. Pick an integer number and find it on the top edge of the third diagram. This point represents the new point E. Draw a line from this point E to the new point E′ and this line, of course, is the second folding line of the new x wave Mobius. Pick another integer for the +yi or −yi component, find it on the top edge line, draw the second fold line, and form the new +yi or −yi wave Mobius. Take these two Mobius and extend their A-B′, B-A′ vertical line out until it counts out, from the 0 line, the number of DNA steps equal to the conjugate number that matches the integer selection. Any flattened Mobius can be folded to tie into increment points set along its top edge. The top edge can be marked off with only the points that represent the DNA step positions, which are, of course, tied to FIGS. 30 to 32. Again pick the number of step points that represent the conjugate number chosen and this time use that as the E point of FIG. 40. This can also be, as pointed out earlier, the A-B′ point. We now have a flattened Mobius with a very long second fold line. This fold line coupled with, and at right angles to, the first fold line forms a very long narrow right triangle similar to those in FIGS. 30 to 32. This Mobius and the other two all start and end at the same point, thus all incorporating the conjugate numbers. In addition the first two work with integer numbers. All three form a very strong system for counting on the DNA molecule.

At 10,000 steps, the wave Mobius is out to its limits. At the limit end, the top and bottom edges still are separated by a single line, the total height of the strip. However, at the starting end, the top edge has folded down to come into the center point, and the strip's height is only from that center point down, one half its original height. The height of the strip of paper at the limit end is double that at the starting end. The next step is to make another identical flattened Mobius strip a tiny bit shorter using opposite hand construction, so that the limit end is in the same position but the starting runs from the center point up to the top edge. Next, the new Mobius strip should be slid inside the old one at the limit end and on top at the starting end. Now, all the very slightly sloping edges match up so two Mobius strips could be joined at these edges, and/or, at any time, they could be divided in half in the same manner they were put together. This suggests that this is the way the two halves are put together. Now each segment of the slightly sloping edge should be numbered; as this is done, a direction arrow should be placed to show the motion of a particle. The first edge segment is 1 for the lower Mobius strip, followed by 2, then 3, then 4, and back to 1 again. Similarly, for the top Mobius strip, the matching edge line is 1, and the arrows are in the same direction, on through 2, 3, and 4 to 1 again. In FIG. 33, there were dual messages on each chain running in the same direction. With two joined Mobius strips, it is possible to have dual arrows or messages running in the same direction along each of the joined edges.

If the joined Mobius strips are made shorter, the cut edges continue to match, but the top fold angle gets steeper. These two Mobius strips, working together off opposite ground lines, support the model of the dual chain spans. By sliding the two Mobius strips inside one another, the result is the perfect match of the Mobius strip to the DNA molecule. This has been accomplished by working with two separate ground lines the Mobius surface strip has made into one continuous edge and not two.

The DNA molecule uses sets of two, or three, hydrogen bridges for the Thymine and Adenine (T and A), and the Cytosine and Guanine (C and G) nucleotide bases, respectively. These bases, (T and A), and (C and G), are shown in FIG. 33. The hydrogen bridges are shown like a series of railroad ties. Additional directional arrows have been added to this diagram.

On the top bridge, the real message from the Adenine molecule leaves the top right nitrogen atom and goes out through its hydrogen atom and across the hydrogen bridge. Another message, the feed-back or imaginary one, leaves the lower hydrogen atom and goes on down through the lower hydrogen bridge.

The dual messages, on the top hydrogen bridge, as the FIG. 33 arrows show, are running in the same direction so that, on the chain that they feed, they will also be running in the same upward direction. On the opposite chain fed by the lower hydrogen bridge, the dual messages are also running in the same downward direction. Obviously, the two sets of messages on chains are running in opposite direction. If a cross sectional cut through both side chains was done on the DNA molecule, there would be four messages. The concept of having the dual messages running in the same direction is tied to FIG. 27.

The foregoing first phase has shown how the 1 to 8 integers are applied to the codons of the DNA ladder. The second phase dealt with the conjugate numbers as applied to the outer chain fringe lines, and the last phase tells how the above application is altered by the four nucleotide letter selection. In general terms, the holographic fringe lines are formed by waves coming through the gaps in the hydrogen bridges and moving across to the outer chain. Each of the three sets of hydrogen bridges contributes to form a one codon length series of fringes. A codon length of fringes on the outer chain rises from the starting point on the first nucleotide base up 3×3.4 A to the top point on the last base. Each of the three hydrogen bridges is set level in a horizontal plane one above the other, but all are firing at the sloping surface rising up in front of them. (When the horizontal diagram of the nucleotide bases is drawn, the outer chain is shown as a horizontal circumferal line which is deceptive.) In order to understand the fringe pattern which represents the conjugate numbers, first the general holographic process must be explained, and then the layout of the base must be looked at.

The DNA molecule uses energy to overcome entropy and process information. Information flows from the environment to the biosystem and back. Energy in all living matter is reduced to quantum mechanics and wave functions. What ties information to wave functions? “In quantum mechanics, for example, the wavelike aspects of matter are described by a mathematical object known as the wave function, which represents everything that is known about the system being described; i.e., it represents the information content of the state.” Davies, The Fifth Miracle, p. 66.

Simply stated, information and waves are a fundamental aspect of biosystems, including those of the DNA molecule. It also should be noted that probability amplitudes are derived from standing waves as noted in the patent application. Waves would be involved in the DNA molecule processing of information, moving across the hydrogen bridges and up and down the side chains. Both the hydrogen bridges and the side chains must have their own means of producing in phase wave motion, and for forming the in-phase holographic interference plates. However, before dealing with these three separate in-phase problems, it is appropriate to review the holographic process.

The holographic processes include a set of two coherent waves coming from a source. One is the reference wave and the other the object wave, together producing an interference pattern which is recorded on a plate. In a photographic process, amplitude information about waves is stored. In the holographic process, both amplitude and phase information about waves is stored. A copying process such as photography that relies on point to point transfer of information is not a complete process and would forego the phase aspect of a biosystem. Phases, that is, time elements, are fundamental to all life processes. The holographic process is commonly thought to involve light, but this is not always the case. As stated in Holography Handbook, Unterseher/Hansen/Schlesinger, p. 366, “We recall that holograms do not necessarily need to be formed with visible light . . . they may be formed in the presence of any wave action. And, ‘it is not the presence of physical waves as such that is needed for making a hologram, but rather an interference pattern, a ratio of harmonic relationships’.” Consequently, it can be seen that the process is far more extensive and basic than just a light application.

The Holography Handbook mentioned above also contains a discussion in which it is stated that holographs must have a recorded interference pattern which is produced by energy, displaying simple harmonic motion passing through standing waves coming from a coherent source. As noted elsewhere herein, the inner source of energy displaying simple harmonic motion is the cycloid type motion of the hydrogen bridge as shown in FIG. 21. The recorder of the interference pattern as noted herein is the holographic plate on the outer chain.

At page 364, the Holography Handbook continues, “The same kind of standing waves can also be generated in a solid: the pattern conforms to the kind of lattice found in a crystal. Since the crystal is vibrating, it can be called an oscillator. If this oscillator is placed near a similar crystal, the two will eventually oscillate in phase forming a tuned resonant system.” This answers the question of how in-phase waves are produced on the hydrogen bridge.

Applying the above quote to the DNA molecule, it is known that the dual and triple hydrogen bridges of the DNA bases will be oscillating in phase with each other. This in-phase oscillation produces either two or three coherent parallel waves, one of which can be used as the object wave and the other as the reference wave. The third is used for the fringe pattern. For the holographic process to take place, there must be an object wave and a reference wave in absolute phase harmony to create useful interference patterns, and there must be a holographic plate to record all this.

The hydrogen bridge is the origin of the three in-phase wave systems, with the third wave leaving the hydrogen bridge and going to the holographic plate.

The simplest explanation of the holographic plate-making process operating on the DNA molecule has one fringe for the integer selection, one for the letter choice, and one for the amino acid selection, each placed very close to parallel to the other varying fringes, all on only a single holographic plate viewed or read from only one angle. This explanation does not allow each nucleotide base to process only its own choice of base. To work with codons shown on FIG. 10 and the other nucleotides, a volume plate must be used.

One of the greatest problems with the holographic process is the loss of the interference patterns following a slight shift in the position of wavefronts. The DNA molecule is extremely narrow; that narrowness permits the source of in-phase holographic waves to be very close to the recording plates, the outer chain of the molecule. Because this distance is so short, it overcomes the possibility of loss of the interference pattern. This very short distance also permits the use of a very minute energy source for the producing and controlling of wave information and overcoming entropy, namely the hydrogen bridge electron. Another advantage of the narrowness of the DNA molecule is that it has a large outer surface area in comparison to its inner volume. This large outer surface area makes it easy for the RNA molecule to read the code at each step along the DNA ladder. Knowing of this ability of the RNA molecule to read information at the outer surface of the DNA molecule, one of the best reasons for its use of the holographic process thus is revealed.

The DNA molecule has up to 10,000 steps; a faulty transfer or reading of 1 or 2 bases can be fatal. For this reason, the reading of the DNA molecule by the RNA molecule should be as close to flawless as possible. The holographic recording plate on the outer chain of the DNA molecule is a volume plate. Unlike a single plate with one set of fringes bounded by two parallel edges, a volume plate is made up of multiple sets of fringes between rows of parallel edges. With the recording plate as a volume plate, multiple identical information is formed on each of the parallel layers of fringes. Because the layer of DNA are in contact with layers of the RNA molecule beneath it, and the fringes are formed by the same set of waves, there is no way that the fringe pattern can shift, or be misread, or produce errors from the DNA molecule to the RNA molecule. The outer layers, the RNA molecule, can then split off with this absolutely correct information.

In the standard explanation in the genetic books, it is always said that the RNA molecule keys into the DNA molecule for the transfer of information. Exactly how that keying is done is never explained. Tying in the holographic process makes it possible to explain how this transfer can be accomplished.

The location of the outer fringes comes from a hydrogen bridge diffraction pattern. The theory expressed herein is that these diffracted waves bouncing off the edges of the holes and the moving electron would be the object waves which form the conjugate number fringe lines. These fringe lines, which would be in-phase and relate to parallel in-phase waves from the hydrogen bridge, come to the side chains. The reference waves adjusted for the fringes would also act as viewing waves carrying the information up and down the length of the DNA molecule.

When one looks at a photograph from different angles, one sees the same picture, but when one looks at a hologram, each angle reveals a different picture. On page 324, the Holographic Handbook says, “Since the hologram will reconstruct with [a wave] at a particular angle, it becomes possible to make another hologram on the same plate which will reconstruct at another angle.” This means that each plate could record the other angles of the other two bases that form the three base codons of the DNA molecule. This means that each plate, viewed from the hydrogen bridge having recorded all three base angles, would appear different. This shows again the fundamental aspect of the holographic process—as is expected, the whole is equal to the sum of the parts, but in a hologram the parts also contain all the information of the whole. Each individual base has a plate with a fringe design that contains the information for that particular base, the other two bases that make up its codon and conjugate the information for the genetic code for that codon.

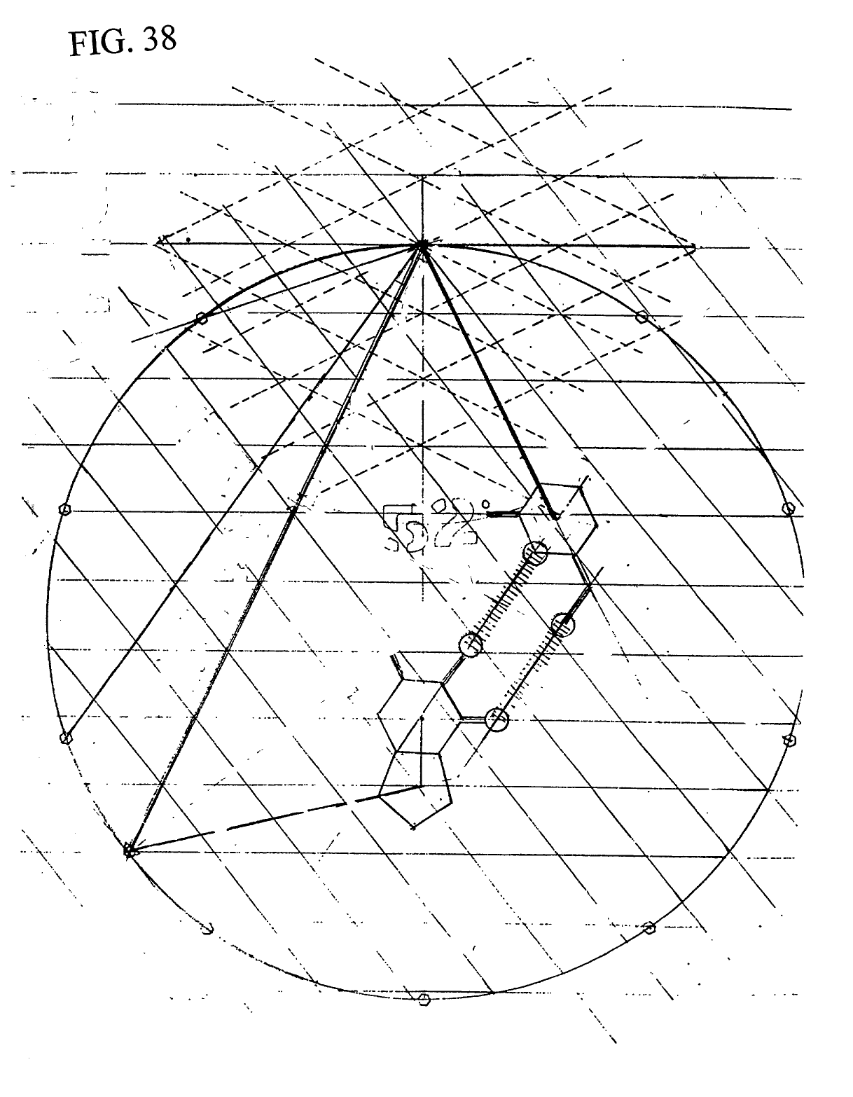

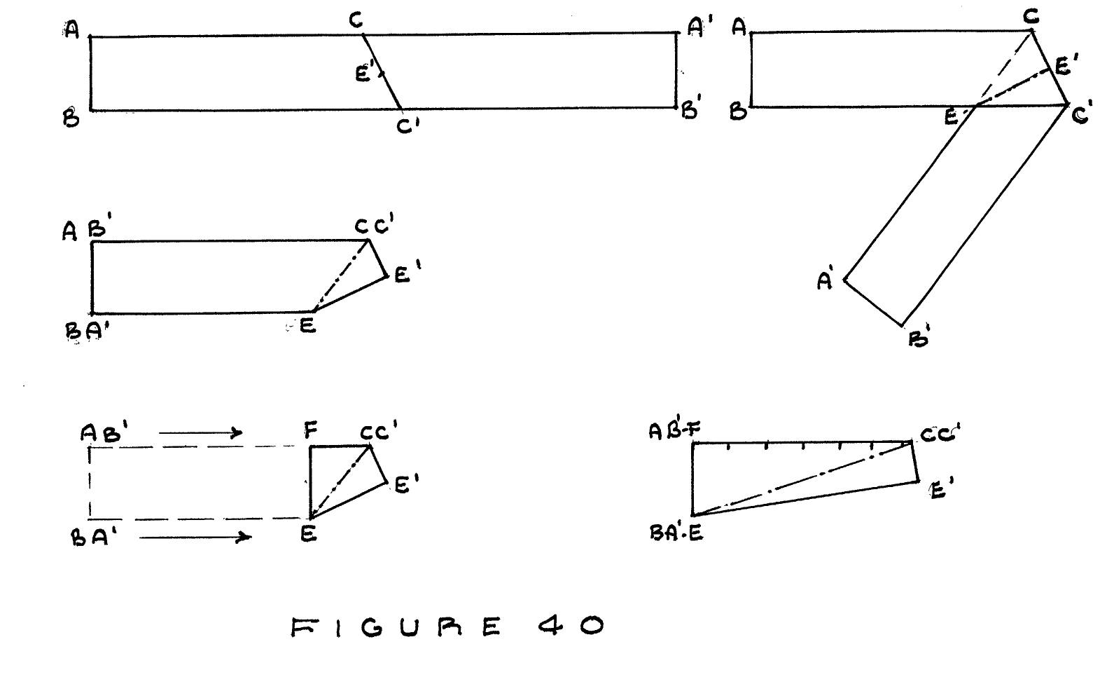

Looking at FIG. 38 in detail, the position of the left and right edges of the front hydrogen bridge opening, particularly with respect to the position of the right edge of the back hydrogen bridge, should be noted. A wave leaving the right edge of the back hydrogen bridge, and touching the right edge of the front bridge, would go straight to the first tangent point, and touching the center electron point of the front bridge, would go to the center of the first and third tangent points. A wave leaving the right edge of the back hydrogen bridge and touching the left edge of the front bridge would go straight to the third base's tangent point. It should be noted that the hydrogen bridges are generally parallel to the line joining the first tangent point to the third tangent point. This parallel relationship changes very slightly for each different nucleotide base. A diffraction pattern using both sides of the opening and the midpoint of the front hydrogen bridge could be distributed over the entire length of this line back to the sloping line surfaces of the three base codon units. However, because this sloping line surface is moving up, the first base relates best to the first step area.

Now, move up to the next base level, where a new FIG. 38 would have its hydrogen bridges turned one notch but would otherwise coincide with the lower FIG. 38. A wave again leaving the right edge of the back hydrogen bridge and touching the right edge or middle of the front bridge would go straight to the area of the lower FIG. 38's second base chord. A wave leaving the left edge of the back hydrogen bridge and touching the left edge of the front bridge would go straight to the end of the lower FIG. 38's second base chord. In other words, the diffraction pattern of the second upper base in FIG. 38 could be used to reinforce the diffraction pattern of the original base. The third base above could also reinforce the patterns of the first two. Put all three levels of FIG. 38 together, and this would be how the diffraction pattern could be used to make the appropriate fringe location. This combined fringe location would represent the conjugate number selections. This fringe would be added to the fringes and wave patterns for the integer numbers.

If the starting point of FIG. 7 were attached to the tangent point of FIG. 38, then after securing these points together, FIG. 7 were wrapped around FIG. 38 so that FIG. 7's entire sloping line from start to finish corresponds approximately to three base units of FIG. 38, in FIG. 38, each base unit is shown with 6.125 Å long chords which is 7.0 Å long on the sloping line. If FIG. 7 is shrunk by a factor of 6, so that its 126 unit long sloping line would correspond to FIG. 38's 21 Å long sloping line, (conjugate 128's actual position is 120 units out from the start), the codon diffraction distribution pattern and the fringes it formed would then also relate to the 126 unit long sloping line of FIG. 7. From FIG. 7, one complex conjugate number from a particular area can be selected. Finally, this 21.0 Å selection can be reduced by a factor of 4/10 to put it on the 8.4 Å total length of the three hydrogen bridges.

As a result, the diffraction distribution pattern just described need only concentrate its heaviest fall on one particular area of FIG. 38 to place one fringe and to pick one particular amino acid. This is how putting FIG. 7 with FIG. 38 and the holographic process produces the genetic code.

Each side chain then has a tangent line that acts as a hologram film. Moving one step up to the next pair of bases, those bases also would have a pair of tangent lines. It can be said that the film plate is a series of tangent surfaces wrapping around the DNA molecule following the curved line of the circle's circumference. Normally in a hologram the object and reference waves form interference areas, the interference area being just in front of and recorded on the plates. The viewer of the hologram would be outside this plate, observing its fringe lines which are used to form the picture of the hologram.

The volume holographic plate has multiple layers, and each layer would be consistent with the others. The preferred waves, coming through the hydrogen bridge must obey Bragg's crystalline scattering rules as applied to volume plates. Each wave would be exactly one wavelength from each other. They would thus be in phase when they passed through each reflecting plate and when they left the entire volume plate construction.

If the DNA molecule used a three-depth plate construction and tied into an RNA molecule which also had a three-depth construction, it could work with a six-deep volume plate. That is assuming first, that the RNA can align itself vertically with a DNA molecule; and second, that it can attach itself so that its set of plates are exactly in phase with the DNA molecule's plates. If this were possible, it would provide an accurate means of transferring information. The RNA molecule would have 3 reflecting plates and be similar in construction to the outer chains of the DNA molecule. For each codon of the DNA, the RNA would have a codon.

When this process is viewed from the top, it should be noted that the waves moving from the nucleotide hydrogen bridges forming the fringe points are tied to the object waves and the reference waves moving along the chain line between the sugar molecules. This association is logical because, at each step of the DNA ladder, the object changes with each different base, and these changes must be transferred to the side chain sugar molecule carrying the object wave, which picks up these changes, and the reference wave, which stays basically unchanged. The structure of the bases changes every step of the DNA ladder which has different sized hydrogen bridges, different angles by which they are joined to the outer uniform chains and different fringe spacing as used on a holographic plate. The DNA uses a structure that gives a uniform answer to the wave processes it uses. Parts of that same structure which change give a constantly varying answer to these wave processes. These two sets of answers are constantly compared. The holographic process will not take place unless a uniform wave is compared to a varying wave.

Viewed from the top, the DNA molecule helix appears as a circle, the circumference of the circle representing the center line of the outer sloping chain of the helix. The nucleotides span across the interior of the circle, running from one side chain to the other. There are ten nucleotides for each complete revolution of the outer chain. The center of these paired nucleotide bases fall generally toward the center of the circle. FIG. 33 is a sample of this top view, showing the four possible nucleotides of the DNA molecule, with arrows and a note “to the chain” at the bottom of each base. What is missing and should have been included are the top view angles between the line “to the chain” and the chains themselves. A line drawn to connect the center of the paired sugar molecules on each chain forms a chord of the circle. On FIG. 38 this line is shown with a dark line connecting the two opposite points on the circle, representing the two paired sugar molecules. The angle between this line and the line “to the chain” going back to the bases is the missing angle. The Thymine base is joined with an angle of 50°; the Adenine base is joined with an angle of 51°; the Cytosine base is joined with an angle of 52°; and the Guanine base is joined with an angle of 54° (Molecular Biology of the Gene by James D. Watson). The sine of those angles are 0.766, 0.777, 0.788, and 0.809 respectively. The averages of these sines is approximately 0.785, corresponding closely to an angle of 52°. As looked at from the top view, 52° is the average angle between the bases and the chord line at the side chains, and is shown on FIG. 38. On each side of this 52° angle are two 64° angles completing the 180° of the tangent line joined to the circumference of the circle. This means a bisecting line of the 52° angle goes back to the center of the circle, as shown on FIG. 38.

When the corresponding sine angle values of each nucleotide or any other numbers are applied to three codon letters in a row, unless the order of the letters is considered, the numbering system is redundant. Put any numbering system on the codon letters, add up the three separate values for a total and any coding system cannot distinguish between, say, TCA, TAC, CTA, CAT, ATC, or ACT. There are 4 sets that have 1 possible letter combination, 12 sets that have 3 possible letter combinations, and 4 that have 6 possible letter combinations, or 20 possible totals. The inventor believes that this redundancy is the reason that nobody has tried to apply a numbering system to the genetic code. This is also the reason that the changing angles of the different hydrogen bridges tied to the conjugate number fringe line placement works.

In other words, the letter selection at each base shifts the conjugate number fringe placement that is sent out to form the total codon fringe placement.

FIG. 38 shows a line from the center of the first step of the outer chain running toward the hydrogen bridges. This line passes through the center point of each of the hydrogen bridges shown with parallel closely placed striped lines.

The center point of each hydrogen bridge connects to the center point of first strand. This lining up of the three points shows that the DNA molecule makes use of the hole in its crystalline structure on the surface of the nucleotides, mainly to bombard the first step. Here there is a constantly changing condition where the hole opens and closes. Obviously, the entire gap has an infinite number of positions between the side atoms, where the waves could fall, but it favors the middle of the first step. It is possible to identify the coherent waves by their amplitude distribution, which also is identical in this case to the pattern obtained from a Fraunhofer diffraction by a single slit or gap (see Fundamentals of Optics, p. 249). These waves plus the wave on the side chain create the holographic pattern. This means that the constructive interference pattern of the holographic waves can be mimicked and coordinated by the constructive wave interference pattern of a wave passing through a slit.

The distribution pattern of this constructive interference is known as a Fourier transform. These transforms are formed when perfectly monolithic waves pass through an opening which is open for only a very short period of time. As in the holographic process, the interference pattern depends on the waves having the same frequency, where the amplitude and the phase are uniform and is possible in this case if the slit and the surface recording the slit are spaced apart. Theoretically, the wave coming from one edge of the slit can be exactly one wavelength farther from the screen than the wave coming from the other edge. This is identical to the Bragg reflecting waves off the parallel planes used by the hologram. In this manner the reflecting process of the waves on the outer chain and the diffracting process of the hydrogen bridge can be related. When the waves land on the screen, they will produce a vibration with a phase difference of π and a zero displacement (Fundamentals of Optics, p. 320). Secondary wavelets also will produce vibrations having a phase difference of π. Wavelets from the next position below the edge wave will cancel those from the next point below the center line, continuing to pair off of all of the wave front.

On FIG. 31, there are 7 laps of gradual spiral between each increase of integers on the 3 steep spirals. The integers increase by 22 with these 7 laps, so each lap is equal to 22/7=3.14285714. This number divided by 1.0004025 equals very close to pi. On FIGS. 13-18, the wave length used was related to pi, but could have been adjusted by 1.0004025. On FIG. 10, the wave lengths used on the side chain and hydrogen bridge were 3.5 Å, and 2.8 Å and on FIGS. 13-18, the wave length used was related to pi, but could be adjusted up to 22/7 or 11/3.5. Using these three mile-marks the basic wave characteristic formula can be written:

(3.5 Å−11/3.5 Å)2.8 Å=(9.8 Å−8.8 Å)=1.000 Å

When a wave on the side chain is reduced by a wave having a 11/3.5 Å length, and the product is multiplied by a wave from the hydrogen bridge, the product is 1. Since both 3.5 Å and 2.8 Å are object waves, then the 11/3.5 Å wave is the uniform wave in the holographic process mentioned in FIGS. 20 to 29. In order for this relationship to work, the size of the uniform wave must go up or down in the same relationship as the side chain waves, and the product must stay equal to 1.0 Å. Dividing both sides of the equation by (3.5 Å−11/3.5 Å) the new equation 2.8 Å=1/(3.5 Å−11/3.5 Å) becomes an inverse relationship where no sets of waves have to be multiplied together to produce a product. 2.8 Å can be changed to 14 Å−11.2 Å, which represents the difference between the hydrogen bridge side chain waves. This difference of wave sizes was shown on the bottom row of numbers in FIG. 10. These differences were also matched with integer numbers positions. Remember on FIG. 10, the first column on the right, 1.75 Å minus 1.4 Å=.35 Å was matched to the integer 1 position. Working with these differences of wave positions and integer number positions, the wave characteristic formula is shown on the left side:

| Wave characteristic formula | |
|---|---|
| chain − bridge = 1/ | Bragg-scattering formula |
| (chain − uniform) = integer | &/2 × sin @′ = d′ |
| 1.75Å − 1.4Å = 1/ | 1 × 11/3.5/2 × .898 = 1.75Å |
| (28.0Å − 8 × 11/3.5Å) integer 1 | |
| 3.5Å − 2.8Å = 1/ | 2 × 11/3.5/2 × .898 = 3.5Å |
| (14.0Å − 4 × 11/3.5Å) = integer 2 | |
| 7.0Å − 5.6Å = 1/ | 4 × 11/3.5/2 × .898 = 7.0Å |
| (7.0Å − 2 × 11/3.5Å) = integer 4 | |
| 14.0Å − 11.2Å = 1/ | 8 × 11/3.5/2 × .898 = 14.0Å |
| (3.5Å − 1 × 11/3.5Å) integer 8 | |

The formula can only be adjusted with geometric changes and not arithmetic changes. Integers 1, 2, 4, and 8 work with the formula, while integers 3, 5, 6, and 7 are missing. Integer 7 can be found be subtracting 1 from 8, 6 is 2 from 8, 5 is 4 plus 1, and 3 is 4 minus 1. As determined, wave patterns can be reduced or supplemented to form peak points. The process used by the DNA molecule is a holographic process. That means that uniform waves are always compared to object waves to determine integer information. Therefore, the integer points on FIG. 10 must be determined by adjusting points 1, 2, 4, and 8, to find points 3, 5, 6, and 7. Now all the integer numbers have been used and related to the uniform waves.

FIG. 10 shows that the small circle 1 point is the first step of the DNA ladder, and is represented by integer 4. The integer 4 equations shown above is cross multiplied:

(7.0 Å−5.6 Å)=1/(7.0 Å−2×11/3.5 Å) changes to (7.0 Å−5.6 Å)(7.0 Å−2×11/3.5)=1

The 7.0 Å above represents the object wave on the side chain and one step on the DNA ladder. 5.6 Å is the object wave on the hydrogen bridge, 2.8 Å over to one side and 2.8 Å back. The 2×11/3.5 Å is the reference wave against which the object wave of the side chain is compared. Remember this formula works with a geometric growth shown above with integer points 1, 2, 4, and 8. To go to the second step on the DNA ladder the equation for step 1 must be repeated. Each step on the ladder involves using the same equation over and over again. (Doubling the 7.0 Å, 5.6 Å, and 2 11/3.5 Å and using them in the equation produces a product equal to 4 not 2).

From the wave characteristic formulas above, pick the integers that go with the conjugate number to be selected. As a simple example, use the conjugate number 80, made up of 8+4i. For the real integer 8 use the formula above 14.0 Å−11.2 Å=1/(3.5 Å−1×11/3.5 Å or in the basic form, 1=2.8 Å(3.5 Å−1×11/3.5 Å). 2.8 Å is the module used on the hydrogen bridge which was maintained by keeping the product equal to 1. Both sides of this equation can be multiplied by 80, the conjugate number, still using 2.8 wave module for the 8 integer. However, as stated before, the formula can only work geometrically, so 80 must be broken down into the geometric numbers 64+16, so:

64=2.8 Å(64×3.5 Å−64×11/3.5 Å)+16=2.8 Å(16×3.5 Å−16×11/3.5 Å)

The same process can be then repeated for the imaginary integer 4 using the formula 7.0 Å−5.6 Å=1/(7.0 Å−2×11/3.5 Å) or in basic form, 1=1.4 Å(7.0 Å−2×11/3.5 Å) 1.4 Å is the wave module this time and again multiply both sides of the equation by 80 in two steps using again 64 and 16.

so,

64=1.4 Å(64×7.0 Å−64×2×11/3.5 Å)+16=1.4 Å(16×7.0 Å−16×2×11/3.5 Å)

In a similar fashion all the other conjugate numbers can be determined by using the wave modules that represent their integers. FIG. 31 made it very clear that 22/7 or 11/3.5 was the module used by codons. The above example shows how the integers are tied to the conjugate numbers on the side chains using wave modules.

From FIG. 38, the angle between the base line and the surface of the plate is 64 degrees. This angle is called the reflecting angle. The side chain of the DNA molecule has a thickness formed of regularly spaced atoms. In dealing with regularly spaced atoms in a crystalline structure, there is a Bragg-scattering pattern. When a set of waves comes in and is reflected off a set of parallel planes formed by uniformly spaced atoms, then the elements of the preferred wave differ from each other by exactly one wavelength. Because the waves are in phase, they produce constructive interference. From Jenkins and White (Fundamental of Optics, p. 665, equation 31 [b]), it can be seen that the Bragg-scattering relationship for these directions is given by the following: &=2 d′ sin @′ where d′ is the distance between reflecting planes, & is the wavelength of the reflected wave, and @′ is the reflecting angle between the parallel planes. The wave length & is 11/3.5 Å related and with an angle of 64 degrees, the sine=0.875. Using these numbers, in the basic formula d′=2×3.142857/2×898=3.5 Å. The distance between reflecting planes that form the volume hologram is 3.5 Å when the wave length of 11/3.5 is raised by a factor of 2. This corresponds directly to the wave characteristic formula for integer 2. The Bragg-scattering formulas with the changing 11/3.5 factors are shown above, matched to the changing integers. The characteristic wave formula came from FIG. 10, the Bragg-scattering formula came from known holographic relationships. The fact that they both directly correspond to each other indicates the correctness of the integer numbers used herein.

The spacing of d or the distance between fringes equals one-half the wavelength or & divided by two, times half the sine of the angle or @, between the reference and object waves (Hansen et al., Holography Handbook, p.325) or d=&/(2 sin[@/2]).

From FIG. 38 the angle between the object and reference waves=52 degrees, so the sine of 26 degrees equals 0.4375×2=0.875. From FIG. 10 the bottom set of numbers shows the hydrogen bridge, side chain wave difference for the four geometric numbers are 0.35 Å, 0.7 Å, 1.4 Å and 2.8 Å, matched to 1.4 Å, 2.8 Å, 5.6 Å, and 11.6 Å hydrogen bridge numbers. Below, the d value is found and this is adjusted by the corresponding bridge number, the product again relating to the integer numbers. The relationship calls in a number of questions, but still, its numbers relate to the integers.

d=0.35 Å/0.875=0.4 Å,1.4 Å−0.4 Å=1

d=0.7 Å/0.875=0.8 Å,2.8 Å−0.8 Å=2

d=1.4 Å/0.875=1.6 Å,5.6 Å−1.6 Å=4

d=2.8 Å/0.875=3.2 Å,11.2 Å−3.2 Å=8

The reference wave pi on FIGS. 20 to 29 took its clue from the hydrogen bridges to form cycloid-sine waves. In the wave characteristic and Bragg-scattering formulas the pi wave has no direct comparison to the hydrogen bridge wave and but is directly compared with the side chain wave. The mathematics of the above formulas indicates that this happened. The wave characteristic formula indicates that there is a direction relationship with the number 1. The crystalline structure of the DNA molecule indicates why this is so.

The four bases along with the entire DNA molecule are a paracrystalline structure. This chemical property helps in analyzing the overall picture. Looking back at FIG. 33, if the hydrogen bridges were completely eliminated, the A and T, C and G molecules would fit snugly together. The parallel lines of the o and h atoms on the one side and the h and n atoms on the other side, noted in FIG. 33, then would merge into one line, to form one line for the four atoms. The top bridge hydrogen atom would be replaced by the upper left side oxygen atom, and the lower hydrogen atom by the lower right side nitrogen atom. A new hexagonal atom grouping would be formed in the middle instead of the bridge, chemically wrong, but visually perfect. This perfect picture of the newly formed complete hexagonal shapes led to the idea that the hydrogen bridges are, in fact, an imperfection in the molecular formations. The idea involves a concept which says simply that the DNA molecule, being a paracrystalline structure, would have lattice type grouping of its atoms, and an imperfection in a lattice structure would be where the molecular pattern would break down. A missing or added atom would be at the break-down point.

Looking at the place where the hydrogen bridge is added as an example of a one-dimensional lattice site with an added atom, and referring to the treatment of this condition in Feynman's Lectures on Physics, there are a whole series of known results. The hydrogen atom in the bridge can be considered the “impurity” atom, and if its energy level is manipulated, there is some probability that its electron would be more predictable. But first it is necessary to establish the different kinds of waves and then see what would happen to a wave on the codons as it comes from this “impurity” site.

At page 13-12 of Volume III of The Feynman Lectures on Physics, Addison-Wesley 1965, Feynman states: “We do not lose any generality if we set the amplitude (&) of the incident wave, [uniform wave] equal to 1. Then the amplitude (B) object wave is, in general, a complex number.” In fact, the most general solution would be a combination of a forward, and a backward added on, (B), waves. Feynman then goes on to say that “what is transmitted beyond the ‘impurity’ site, the transmitted wave (Y) is just the original incident wave (1) with an added wave (B) equal to the reflected wave. This being true only when you go through a single atom” (such as the single hydrogen atom). So (Y), the transmitted wave, is the message that goes out as the code and it works by considering the hydrogen atom as an “impurity” site.

Now a picture of the hydrogen dual messages is presented, but is necessary to consider how to turn the corner onto the chains. They, too, as mentioned before, must carry the dual messages. It can be said simply that there must be some sort of reflecting surface or process that put dual (Y) messages to become the side codons. There is another hydrogen atom on the side chains, not shown in FIG. 33, which perform this reflecting chore. So now it can be said, about the (B) and the (1) forming the (Y), that all three relate to the side chain. At page 13-12 of the above-referenced Volume III of The Feynman Lectures on Physics, Feymnan goes on to say: “Remember, though, B and Y are complex numbers and that the number of particles (or rather, the probability of finding a particle) in a wave is proportional to the absolute square of the amplitude. In fact, there will be conservation of electrons only if B²+Y²=1”.

Working with imaginary exponents for the moment, it should be noted that changing i to −i is called taking the complex conjugate. If this is done throughout an equation, it does not change the results. Taking the complex conjugate of 10^{i(s)}=x+iy, yields 10^{-i(s)}=x−iy. We know from the general rules of exponents that 10^{-i(s)}=1/(10^{i(s)}. Then x−iy=1/(x+iy). These numbers are the reciprocal of each other. Multiplying each side by x+iy, the left side is (x−iy)(x+iy)=x+iy/x+iy, or x²+y²=1. Put more simply, 10^{i(s)}×10^{-i(s)}=(x+iy) (x−iy)=10⁰=1. To make up the formula (x+iy) (x−iy) three curves, the x, iy, and −iy curves are necessary. Changing x to −x yielded the same results had the negative cosine curve (−x) been used instead of (x)=(−x+iy) (−x−iy)=1, x²+y²=1. This is a formula used in the genetic process. Squaring both the imaginary and the real parts of the complex numbers is what was done in FIG. 7 to get the genetic code.

It should be remembered that, in the last diagram of FIG. 40 when the short side of the wave right triangles was used for a series of reciprocal numbers at point CC, these numbers were the inverse of the long BA′-E numbers. Consequently, if the long number reads 3, the small number would read ⅓, etc. These numbers started at 1 and reduced down to zero. As x+iy and 1/(x−iy) are reciprocal, if x+iy represents the numbers on the top chain, 1/(x−iy) would be the numbers on the lower chain. Multiplying both the top and bottom chain numbers by x−iy makes the top chain (x+iy) (x−iy), or x²+y^{2,} and the bottom chain

$$\frac{( x - iy )}{( x - iy )}$$

becomes 1. Obviously since this stays at 1 no matter what the numbers are, this is part of the metro-tone of the system. Now a mathematical number system for the process is starting to form that can be clarified using geometry.

Earlier in the detailed description of a preferred embodiment of the present invention, reference was made to the use of a computer to generate complex conjugates and codon sequences. It will be appreciated that such computer assistance would be invaluable not only in generating DNA sequences, but also in predicting such sequences, based again on criteria such as the frequency of occurrence of the various codons in nature. Further applicability of the invention to biologically-based computational apparatus also are within the inventor's contemplation, based on the extensive numerical relationships detailed herein. The holographic nature of the DNA molecules, and the resulting precise fringe characteristics also facilitate the prediction and generation of DNA sequences because of the required precise distance relationships, based on the frequency of occurrence of the various amino acids in nature. The holographic nature of the DNA molecules also yields intriguing results when considering the use of such molecules, both to store and to transmit digital data, either in the form of a memory or in the form of a computational device.

While the invention has been described in detail above with reference to some presently preferred embodiments, various modifications within the scope and spirit of the invention will be apparent to those of working skill in this technological field. Accordingly, the invention should be construed as limited only by the appended claims.

What is claimed is:

1. A computer-implemented method of generating DNA sequences comprising:
identifying a plurality of distances on a hydrogen ladder between two strands of DNA;
expressing said distances as lines in a plane, such that said lines have a slope that is substantially equal to the pitch of a DNA helix;
equating said distances with codons;
assigning said distances to amino acids in one-to-one correspondence with molecular structures of said amino acids, and in one-to-one correspondence with statistical frequency of occurrence of said amino acids in nature; and
generating said DNA sequences as a result of the outcome of said equating and assigning steps.

2. A method as claimed in claim 1, wherein said assigning comprises, based on relative prevalence of codon letter combinations in nature, assigning said codon letter combinations to codon positions in said strands of DNA.

3. A method as claimed in claim 2, wherein said codon letter combination assigning comprises:
defining an origin;
dividing said codons into first, second, and third components;
defining first, second, and third spiral paths extending from said origin; and
placing said first components on said first spiral path, said second components on said second spiral path, and said third components on said third spiral path,
wherein positions of said first through third components along said first through third spiral paths correspond to positions of said components on a DNA helix.

4. A method as claimed in claim 2, further comprising:
matching said codons to an identical number of complex conjugate combinations, wherein a complex conjugate number is expressed as x+iy, 1≦x, y≦8;
placing ones of said amino acids into respective groups as a function of common functions of said amino acids, and comparing said groups to said complex conjugate combinations; and
in accordance with said matching and placing steps, and also based on said relative prevalence of codon letter combinations in nature, assigning said codon letter combinations to codon positions in said strands of DNA;
wherein there are 64 of said complex conjugate combinations, said matching step placing said 64 complex conjugate combinations into four types of groups, a first group type containing a single group having three of said complex conjugate combinations, a second group type containing five groups each having one of said complex conjugate combinations, a third group type containing eight groups each having four of said complex conjugate combinations, and a fourth group type containing 12 groups each having two of said complex conjugate combinations.

5. A method as claimed in claim 4, wherein said first through fourth group types contain a total of 26 groups, said placing step comprising reducing said 26 groups to 20 groups, and assigning each of said 20 groups to a respective amino acid.

6. A method as claimed in claim 1, further comprising modeling movement of electrons across said hydrogen bridges between two strands of DNA based on definitions of cycloid paths created with circles of unit diameter.

7. A method as claimed in claim 6, wherein said cycloid paths correspond to reciprocal paths on said hydrogen bridges, between said strands of DNA.

8. A method as claimed in claim 1, further comprising modeling movement of electrons across said hydrogen bridges between two strands of DNA based on probability amplitudes indicative of likely positions of said electrons, said probability amplitudes being defined as a function of a wave nature of said electrons.

9. A computer-implemented method of predicting DNA sequences comprising:
identifying a plurality of distances on a hydrogen ladder between two strands of DNA;
expressing said distances as lines in a plane, such that said lines have a slope that is substantially equal to the pitch of a DNA helix;
equating said distances with codons;
assigning said distances to amino acids in one-to-one correspondence with molecular structures of said amino acids, and in one-to-one correspondence with statistical frequency of occurrence of said amino acids in nature; and
predicting said DNA sequences as a result of said equating and assigning.

10. A method as claimed in claim 4, wherein said codon letter combination assigning comprises:
defining an origin;
dividing said codons into first, second, and third components;
defining first, second, and third spiral paths extending from said origin; and
placing said first components on said first spiral path, said second components on said second spiral path, and said third components on said third spiral path,
wherein positions of said first through third components along said first through third spiral paths correspond to positions of said components on a DNA helix.

11. Computer-implemented apparatus for generating DNA sequences, comprising:
means for quantifying a plurality of distances on a hydrogen ladder between two strands of DNA;
means for equating said distances with codons;
means for assigning said distances to amino acids, said means for assigning comprising in turn the following:
means for placing ones of said amino acids into respective groups based on common functions of said amino acids; and
means for assigning said codon letter combinations to codon positions in said strands of DNA based on outputs of said placing means, and also based on relative prevalence of said codon letter combinations in nature;
said apparatus further comprising means for generating said DNA sequences in response to outputs of said equating means and said codon letter combination assigning means.

12. Apparatus as claimed in claim 11, further comprising means for modeling movement of electrons across said hydrogen bridges between two strands of DNA based on probability amplitudes indicative of likely positions of said electrons, said probability amplitudes being defined as a function of a wave nature of said electrons.

13. A computer-implemented method of generating DNA sequences comprising:
i) using a holographic property of DNA sequences to identify regularly spaced base positions along the sequences; and
ii) placing amino acids in the respective base positions, each of the amino acids having a codon letter combination associated with it, each of the codon letter combinations having an associated angular variation of a connection of its respective amino acid to a DNA helix.
